(19) United States
(12) Patent Application Publication
(10) Pub. No.: US 2025/0281295 A1
(43) Pub. Date: Sep. 11, 2025

(54) SYSTEMS AND METHODS FOR CAVAL FLOW BALANCING VIA VALVE-LIKE MECHANISMS

(71) Applicant: EDWARDS LIFESCIENCES CORPORATION, Irvine, CA (US)

(72) Inventors: Scott Louis Pool, Laguna Hills, CA (US); Arvin T. Chang, Yorba Linda, CA (US); Yaeer E. Lev, Torrance, CA (US); Daniel M. Harps, Trabuco Canyon, CA (US); Amir Yousef Sajjadi, Aliso Viejo, CA (US)

(73) Assignee: EDWARDS LIFESCIENCES CORPORATION, Irvine, CA (US)

(21) Appl. No.: 19/215,942

(22) Filed: May 22, 2025

Related U.S. Application Data
(63) Continuation of application No. PCT/US2023/083624, filed on Dec. 12, 2023.
(60) Provisional application No. 63/387,924, filed on Dec. 16, 2022.

(51) Int. Cl. A61F 2/24; A61B 5/00; A61B 5/0215; A61F 2/48

(52) U.S. Cl. CPC A61F 2/2475; A61B 5/02152; A61B 5/4836; A61B 5/6862; A61F 2/2418; A61F 2/482; A61F 2210/009

(57) ABSTRACT

A device may include a control element coupled to a valve disposed in an outer frame. The control element is configured to manipulate the valve between an unrestricted blood flow state and a restricted blood flow state. A device may include an actuation device including a linear actuator coupled to a control element of an implantable device, and a first magnet configured to induce rotation of the linear actuator. A control device communicatively coupled to the linear actuator includes a second magnet configured to generate a changing magnetic field pole direction to cause rotation of the first magnet either to tension the control element to position a valve of the implantable device in a restricted blood flow state or to release tension in the control element to position the valve in an unrestricted blood flow state.

### CROSS REFERENCE TO RELATED APPLICATIONS

This application is a continuation of International Application No. PCT/US2023/083624, filed Dec. 12, 2023, which claims the benefit of U.S. Provisional Application Ser. No. 63/387,924, filed Dec. 16, 2022, the contents of each of which are herein incorporated by reference.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of medical devices and procedures, and more specifically to the field of blood flow management in blood vessels.

### BACKGROUND

Chronic kidney disease (CKD) is a common comorbidity with many patients also suffering from chronic Heart Failure (HF). HF patients may also have elevated right atrium pressure, which may impair kidney function. In HF patients with elevated right atrium pressure, the kidneys may attempt to perform a diuresis process, but such a process may be difficult to perform efficiently due to the elevated pressure. For example, elevated right atrium pressure may hinder the ability of the kidneys to drive forward the flow of blood for accomplishing proper and efficient diuresis. Such unbalanced pressure coupled with the typical poor kidney efficiency of CKD patients may lead to an unending cycle of fluid overload for a person, which may result in an increase in congestion and heart failure admissions to the hospital.

### SUMMARY

In some aspects, the techniques described herein relate to a system for modulating blood flow through a blood vessel, the system including: an implantable device including: an outer frame, a valve disposed in the outer frame, a control element coupled to the valve, wherein the control element is configured to manipulate the valve between an unrestricted blood flow state and a restricted blood flow state, and a sensor coupled to the implantable device; and an actuation device including: a linear actuator coupled to the control element, a power source, and a microcontroller electrically coupled to the power source, the linear actuator, and the sensor, wherein the linear actuator is configured to tension the control element to position the valve in the restricted blood flow state in response to a first pressure state sensed by the sensor, and wherein the linear actuator is configured to release tension in the control element to position the valve in the unrestricted blood flow state in response to a second pressure state sensed by the sensor.

In some aspects, the techniques described herein relate to a system for modulating blood flow through a blood vessel, the system including: an implanted actuation device including: a linear actuator coupled to a control element of an implantable device, and a first magnet configured to induce rotation of the linear actuator; and a control device communicatively coupled to the linear actuator, wherein the control device includes: a second magnet configured to generate a changing magnetic field pole direction to cause rotation of the first magnet, a power source, and a microcontroller electrically coupled to the power source, the second magnet, and a sensor communicatively coupled to the implantable device, wherein, in response to a first pressure state sensed by the sensor, the microcontroller is configured to cause rotation of the second magnet in a first direction to induce rotation of the first magnet, thereby causing the linear actuator to tension the control element to position a valve of the implantable device in a restricted blood flow state, and wherein, in response to a second pressure state sensed by the sensor, the microcontroller is configured to cause rotation of the second magnet in a second direction to induce rotation of the first magnet, thereby causing the linear actuator to release tension in the control element to position the valve in an unrestricted blood flow state.

In some aspects, the techniques described herein relate to an implantable device configured to regulate blood flow in a blood vessel, the implantable device including: an outer frame; a valve disposed in the outer frame; and a control element coupled to the valve, wherein the control element is configured to manipulate the valve between an unrestricted blood flow state and a restricted blood flow state.

In some aspects, the techniques described herein relate to a system for modulating blood flow through a blood vessel, the system including: an implantable device including: an outer frame, a valve disposed in the outer frame, and a control element coupled to the valve, wherein the control element is configured to manipulate the valve between an unrestricted blood flow state and a restricted blood flow state; and an actuation device including: a pump fluidly connected to a reservoir, a chamber including a first portion and a second portion, a manifold fluidly connected to the pump, the reservoir, and the chamber, wherein the manifold includes at least one port configured to fluidly connect the reservoir to the first portion of the chamber, and a piston coupled to the control element and configured to move between a restricted blood flow position and an unrestricted blood flow position within the chamber, wherein: the piston is configured to move to the restricted blood flow position when a fluid is flowed from the reservoir through the manifold into the first portion of the chamber, and the piston is configured to return to the unrestricted blood flow position when the fluid is evacuated from the first portion of the chamber.

In some aspects, the techniques described herein relate to an actuation device configured to regulate blood flow through an implantable device positioned in a blood vessel, the actuation device including: a pump fluidly connected to a reservoir; a chamber including a first portion and a second portion; a manifold fluidly connected to the pump, the reservoir, and the chamber, wherein the manifold includes at least one port configured to fluidly connect the reservoir to the first portion of the chamber; and a piston coupled to a control element of an implantable device and configured to move between a restricted blood flow position and an unrestricted blood flow position within the chamber, wherein: the piston is configured to move to the restricted blood flow position when a fluid is pumped from the reservoir through the manifold into the first portion of the chamber, and the piston is configured to return to the unrestricted blood flow position when the fluid is evacuated from the first portion of the chamber.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates one embodiment of blood flow balancing using an implantable device in a blood vessel.

FIG. 1B shows a schematic of an embodiment of a system for modulating blood flow through a blood vessel.

FIG. 2A illustrates an embodiment of an actuation device configured to cause substantially unrestricted blood flow through an implantable device.

FIG. 2B illustrates an embodiment of an actuation device configured to cause restricted blood flow through an implantable device.

FIG. 2C illustrates an embodiment of an actuation device configured to cause substantially unrestricted blood flow through an implantable device.

FIG. 2D illustrates an embodiment of an actuation device configured to cause substantially unrestricted blood flow through an implantable device.

FIG. 2E illustrates an embodiment of an actuation device configured to cause restricted blood flow through an implantable device.

FIG. 3A shows a schematic of an embodiment of a system for modulating blood flow through a blood vessel.

FIG. 3B shows a schematic of an embodiment of a system for modulating blood flow through a blood vessel.

FIG. 4A illustrates an embodiment of a blood flow balancing implant controllable by a linear actuator.

FIG. 4B is a zoomed in side-view of the linear actuator of FIG. 4A.

FIG. 4C is a zoomed in cross-sectional view of the linear actuator of FIG. 4A.

FIG. 5A illustrates an embodiment of a system for modulating blood flow through a blood vessel, the system allowing substantially unrestricted blood flow.

FIG. 5B illustrates the embodiment of FIG. 5A in a substantially restricted blood flow configuration.

FIG. 6A illustrates an embodiment of a system for modulating blood flow through a blood vessel, the system substantially restricting blood flow.

FIG. 6B illustrates the embodiment of FIG. 6A in a substantially unrestricted blood flow configuration.

FIG. 7A illustrates an embodiment of a system for modulating blood flow through a blood vessel, the system allowing substantially unrestricted blood flow.

FIG. 7B illustrates the embodiment of FIG. 7A in a substantially restricted blood flow configuration.

FIG. 7C illustrates an embodiment of a valve or valve portion.

FIG. 8 illustrates a schematic representation of portions of a subject that may include a flow modulating device implanted therein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable modulating and/or balancing of blood flow through a blood vessel. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body.

The examples presented herein may relate to providing devices, methods, and/or methods of treatment (MOTs) for modulating and/or otherwise managing blood flow to or through one or more blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as the Superior Vena Cave (SVC) and the Inferior Vena Cava (IVC), or related vessels. Managing blood flow through the SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the SVC and/or IVC, resulting in improved kidney perfusion and function.

In some examples, the devices, methods, and/or MOTs described herein may be utilized to solve a technical problem of unwanted pressure increases in the right atrium in patients that have chronic kidney disease (CKD) and/or heart failure (HF). For example, patients with CKD and/or HF may exhibit reduced kidney function when pressure in the right atrium of the heart is above a predefined pressure threshold. The predefined pressure threshold may be used as a basis to determine whether a patient is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above. For example, each of the devices described herein may be used to decrease pressure within one or more vessels to avoid right atrium pressure increases and/or pressure variations. In particular, the devices, methods, and/or MOTs described herein can be used to reduce and maintain low pressure in the right atrium, which provides a technical effect of enabling the kidneys to more effectively filter blood.

In addition, the devices, methods, and/or MOTs described herein can solve a further technical problem of accumulation of blood in the venous system. For example, the devices described herein may be used to reduce the accumulation of blood in the venous system, which can provide an advantage and technical effect of ensuring that pressure is not increased in the SVC and/or IVC. Such devices can advantageously eliminate excessive hospital readmissions and/or can provide for a long-term blood flow management therapy, improving both quality of life and overall survival rates and with a lower cost to a healthcare system.

Furthermore, the devices, methods, and/or MOTs described herein can be used to solve a further technical problem of regulating blood flow return, thus further mitigating pressure build-up in the right atrium. The examples described herein can perform blood flow management actively and/or passively to assist in reducing and/or maintaining right atrium pressures to a relatively low pressure even when a surge in blood volume occurs in one or more vessels of the venous system.

Although restricted and unrestricted flow states or restricted and unrestricted valve positions are described herein, it is within the scope of the present disclosure that any number of intermediate positions or states are contemplated and included herein, whether or not expressly indicated.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve-based system (e.g., with or without a manifold), hydraulics, pneumatics, optical actuators, thermal actuators, and/or other actuators using electrical or inductive power.

In some implementations, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include a pump fluidly connected to a reservoir; a chamber having a first portion and a second portion; a manifold fluidly connected to the pump, the reservoir, and the chamber; and a piston coupled to a control element of a flow modulating device. The manifold may include at least one port that fluidly connects the reservoir to the first portion of the chamber. The piston can move between a restricted blood flow position and an unrestricted blood flow position within the chamber or any position therebetween for intermediate blood flow restriction positions. For example, the piston may move to the restricted blood flow position when a fluid flows from (or is pumped from) the reservoir, charged by a pump, through the manifold into the first portion of the chamber. The piston can return to the unrestricted blood flow position when the fluid is evacuated from the first portion of the chamber. In some embodiments, the manifold is fluidly connected to a second portion of the chamber through a second port. In such embodiments, the piston can move to the unrestricted blood flow position when the fluid enters the second port from the reservoir through the manifold, thereby causing the valve of the flow modulating device to move to the unrestricted blood flow state. In some embodiments, the fluid is evacuated from the second portion of the chamber through the second port when the piston is in the restricted blood flow position. In some implementations, the at least one port further fluidly connects the first portion of the chamber to the pump through the manifold. For example, the at least first port is fluidly connected to the pump through the manifold to evacuate the fluid from the first portion of the chamber thereby moving the piston to the unrestricted blood flow position. In some examples, the piston is a spring-based piston. For example, the spring-based piston can automatically return to the unrestricted blood flow position when the fluid is evacuated from the first portion of the chamber.

In any of the embodiments described herein, an active mechanism may include a linear actuator coupled to a control element of the flow management device. The linear actuator tensions the control element to position the valve of the flow management device in a restricted blood flow state. Alternatively, the linear actuator releases tension in the control element to position the valve in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some embodiments, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, decreasing a temperature of a thermal sensitive fluid (e.g., via a heat source, changes in body temperature, etc.) causes the piston to compress the fluid to tension the control element to move the valve into a restricted blood flow state. Alternatively, increasing the temperature of the thermal sensitive fluid causes the piston to decompress the fluid to release tension in the control element to move the valve to an unrestricted blood flow state.

As used herein, the term “passive” with respect to blood flow management may represent operations carried out by any of the devices described herein using passively induced movement. For example, passively moving a portion of the devices described herein may include the use of manual pullwires (e.g., sutures, actuation wires/cords, etc.), anatomy responses (e.g., changes in vessel inner diameter, intra-vessel pressure, etc.), blood movement, or the like.

In some examples described herein, a passive mechanism can include a control element, such as a rigid push or pull member (e.g., a rod or rigid wire), a flexible tension member, a flexible wire, a suture, a string, a cable, or the like. One or more control elements can be coupled to a body to be moved (or actuated), such as one or more leaflets, flaps, valves, or valve portions (e.g., used to restrict blood flow through a blood vessel) and to a frame (or other component). For example, the control element can be coupled to a frame that moves in response to an external force (e.g., an increase in blood pressure). In some examples, the control element is coupled to a helical tube that expands and contracts with a blood vessel in which it is positioned, and the movement of the helical tube moves the control element. In some examples, a control member can be used to actuate the valve in response to the movement of the frame (or helical tube) due to a change in blood pressure within a blood vessel.

In some examples described herein, a passive mechanism can include a spring, or elastic member (such as a flexible commissure). One or more springs or elastic members can be coupled to a body to be moved (or actuated), such as one or more leaflets, flaps, valves, or valve portions (e.g., used to restrict blood flow through a blood vessel) and to a frame. For example, a spring can be used to bias the leaflet, flap, or valve in an initial position (e.g., under relatively lower blood pressure conditions). An externally applied force can cause the leaflet, flap, or valve to move and extend the spring (e.g., under relatively higher blood pressure conditions, for example wherein increased blood flow applies pressure against the leaflet, flap, or valve). In the absence of the applied force, the spring can recompress, thereby bringing the leaflet, flap, or valve back to its initial position.

In some examples described herein, a passive mechanism can include more than one mode of operation. For example, a body (or member or component) of a blood flow regulator (or restrictor) described herein can move in a particular way in response to a first externally applied force, and then the body (or member or component) of a blood flow regulator (or restrictor) described herein can move in a different way in response to a second externally applied force. In some cases, the first externally applied force and the second externally applied force are applied from the same external force (e.g., blood pressure), with different quantitative ranges. For example, a leaflet, flap, or valve (e.g., used to restrict blood flow through a blood vessel) can move in a first mode in response to an increase in blood pressure within a first blood pressure range, and move in a second mode in response to an increase in blood pressure within a second blood pressure range that is different (e.g., higher) than the first blood pressure range. The first mode can be that an end of the leaflets, flaps, or valve portions moves towards one another to further restrict blood flow within the blood vessel, and the second mode can be that the leaflets, flaps, or valve portions prolapse, thereby moving away from one another to increase the blood flow within the blood vessel.

In some examples described herein, a flow modulating device (e.g., a flow restrictor) described herein can include more than one passive mechanism. For example, a flow modulating device can contain a first passive mechanism that can move in a first mode in response to an increase in blood pressure within a first blood pressure range, and a second passive mechanism that can move in a second mode in response to an increase in blood pressure within a second blood pressure range that is different (e.g., higher or maximum) than the first blood pressure range. The first passive mechanism can include leaflets, flaps, or valve portions that move towards one another to further restrict blood flow within the blood vessel, and the second passive mechanism can include an inner valve that can move (e.g., axially) to open additional channels through the flow regulator device to increase the blood flow. In some cases, the inner valve can be biased using a passive element such as one or more springs or spring like elements, such that it will return to its initial position after the blood pressure decreases (e.g., back into a first blood pressure range).

Any of the implantable or flow modulating devices described herein may be coated with a polymer (e.g., silicones, poly(urethanes), poly(acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly (ethylene terephthalate), while the nonwoven fabrics may be made from expanded poly(tetrafluoroethylene). Some textiles may also or alternatively include silk or silk-based materials.

Further, any of the pull wires, sutures, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating components described herein. Such stents may include an assembly with strut members interconnected by joints that form a series of linked members that result in a hollow tube-shaped frame. The stents may be positioned and/or repositioned within a blood vessel to introduce or remove flow modulating devices or components including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device or component. For example, a stent or stent portion may surround a portion of a flow modulating device or component to ensure the device remains in a specified position in a blood vessel. In some examples, the stent surrounds the flow modulating device or component entirely. In some examples, the stent surrounds the flow modulating device or component and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices or components described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

### Overview

Various embodiments of systems and devices for modulating blood flow through a blood vessel are contemplated herein. The embodiments described herein have been contemplated for use in a patient/user having chronic heart failure and/or chronic kidney disease, but may be used in any vessel needing flow modulation therethrough.

The embodied systems may include an implantable device, further including an outer frame, a valve disposed in the outer frame (e.g., stent), and a control element (e.g., a wire, cable, suture, etc.). The control element manipulates the valve between an unrestricted blood flow and a restricted blood flow (and between any one or more intermediate positions).

The systems described herein may include an actuation device, either active or passive. The actuation device can include a pump, a reservoir, and a chamber separated into a first portion and a second portion by a piston. The first portion of the chamber may be ported in such a way that the piston manipulates the control element to a restricted blood flow position when moved by fluid flowed into the first portion of the chamber or to an unrestricted blood flow position when fluid is evacuated from the first portion of the chamber. The actuation fluid of the actuation device may be a gas or a liquid, i.e., the actuation device may be hydraulically or pneumatically actuated. The actuation fluid may also be rechargeable either by pressurizing a gas or by creating stored mechanical energy (e.g., pumping liquid into an elastic bladder or into a pressure tank). The actuating device can include a power source responsible for powering the pump or any other power requiring devices. This stored power source may be electrical and used to power an electromechanical pump. Other embodiments may include a pump powered by an anatomy of the user. An anatomically powered pump may include a pump actuated by the breathing of the user or changes in blood pressure in a vessel of a user. For example, the pump (of the actuation device) or frame (of the implantable device) may be compressed during the expansion of the user's diaphragm (i.e., during exhalation of the user) and relaxed during the reduction of the user's diaphragm (i.e., during inhalation of the user). Further, for example, the pump or frame may be compressed during high blood pressure or decompressed or less compressed during low blood pressure. With properly placed check valves and a re-expanding bladder, fluid may be pumped and/or pressurized. Additionally, an external pump may be used with subcutaneous access to system described herein. The external pump may be autonomous and self-powered, or manual and powered by the patient or healthcare professional.

Embodiments of the systems described herein may include a second portion of a chamber with a port for the entrance and evacuation of fluid. The second chamber portion port may aid in the evacuation of fluid from the first chamber portion when fluid is flowed into the second chamber portion. The resulting force on the piston may be used to drive the fluid from the first chamber portion. The piston drives fluid from the second chamber portion when fluid is flowed into the first chamber portion. The driven fluids of both the first chamber portion and the second chamber portion are driven into the inlet of the pump or into a reservoir for later usage, or, in further embodiments, vented into the anatomy.

Directing the fluid to and from the first chamber portion port, and, in some embodiments, the second chamber portion port, may be a manifold. For example, the manifold may include an input element (e.g., slider or other mechanism controllable by a control device). In positioning an input element of the manifold in a first position, fluid communication with the pump and reservoir may be established with the first chamber portion port, and, in some embodiments, the second chamber portion port. An example position of the input element may include one in which the pump is in communication with the first chamber portion and the reservoir is in communication with the second chamber portion. The example position of the input element may be the selected position for restriction of blood flow. Another example position of the input element can include one in which the pump is in communication with the second chamber portion and the reservoir is in communication with the first chamber portion. This second described position may be selected for relieving the blood flow restriction. Depending on the measurements of one or more sensors, the input element may manipulate the manifold to achieve any position between, and including, the restricted blood flow position and the unrestricted blood flow position.

Further embodiments can include a piston spring disposed in the second chamber portion. This piston spring, biased in the unrestricted direction of the piston, may be used to return the piston to the unrestricted blood flow position and evacuate fluid from the first chamber portion, thus eliminating or reducing the need for the second chamber portion port. It is further contemplated that embodiments may not include a second chamber port or a piston spring. These embodiments may use suction from the pump or draining to evacuate fluid from the first chamber portion and return the piston to the unrestricted blood flow position.

Embodiments of the actuation device may include one or more one-way valves. A one-way valve may be a check valve, a non-return valve, a reflux valve, a retention valve, or foot valve. For example, the one-way valve can be a check valve. A one-way valve may be installed on the inlet of the pump and a one-way valve may be installed on the outlet of the pump to ensure correct flow direction. In some implementations, regulating flow direction may be used when fluid pressure is present on the outlet of the pump.

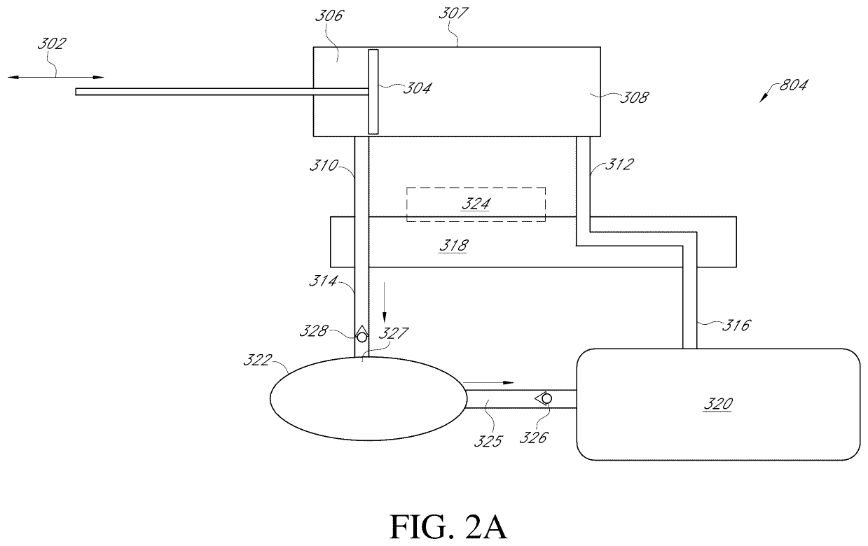

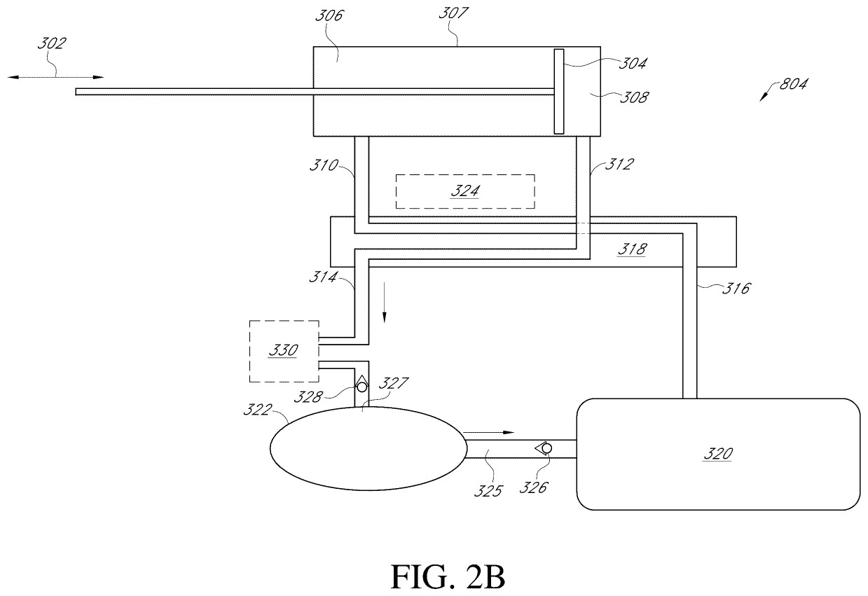

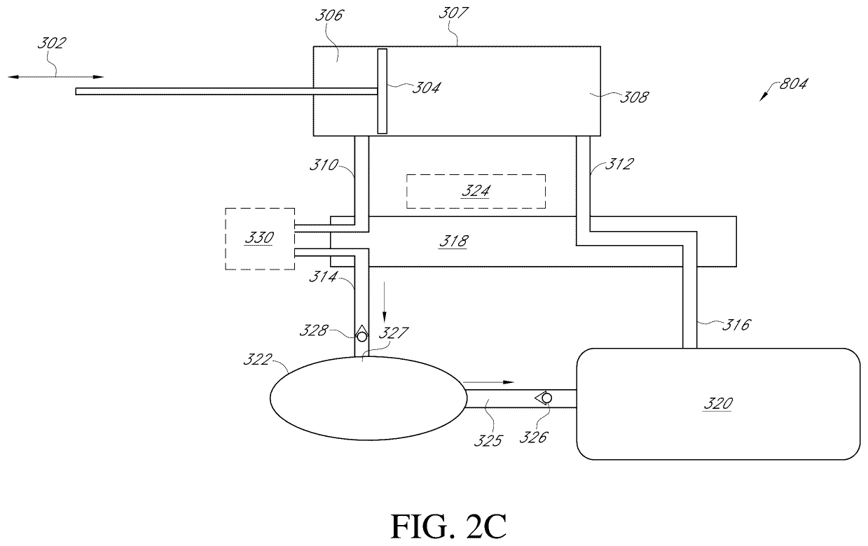

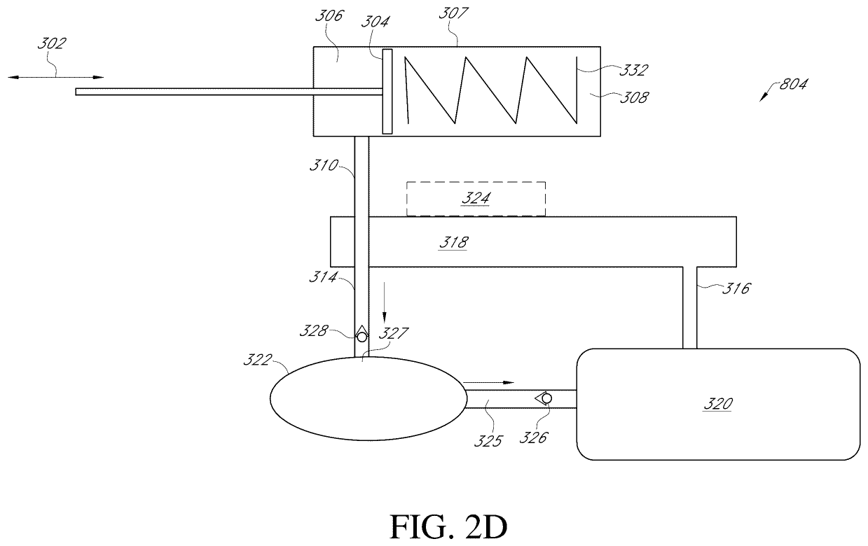

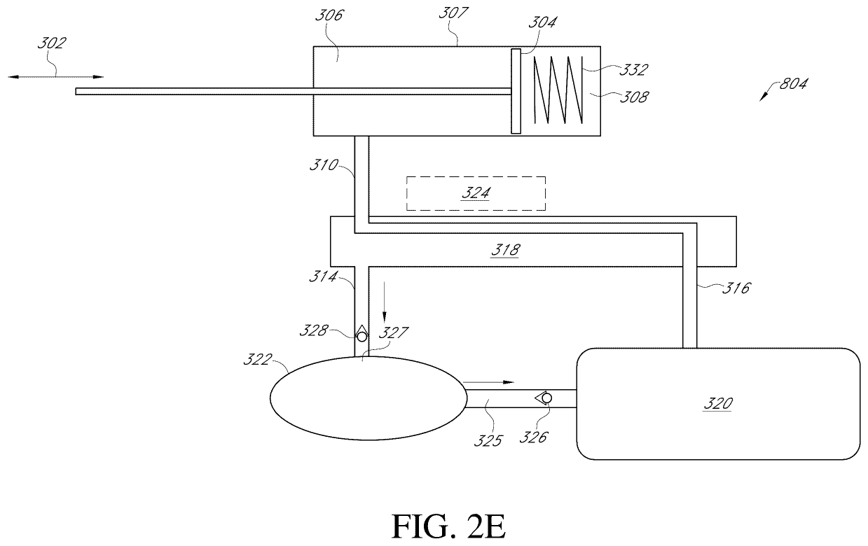

The configuration above includes contemplation of the reservoir installed on the output line of the pump. In this way, a gas can be pumped into the reservoir and pressurized. Once pressurized, the gas can be used for the movement of the piston at any point while the pump is or is not active. The same stored potential may be created with a liquid and a reservoir with an elastic construction or with a spring (or compressed gas) collapsed bladder. Further embodiments are contemplated including those with a reservoir on the input side of the pump or embodiments with reservoirs on both the output and input sides of the pump. Benefits of an optional reservoir 330 (shown in FIGS. 2B and 2C) on the input side of the pump may include auxiliary fluid volume to avoid vacuum effects in the intake tubing. Additionally, and in some embodiments, the optional reservoir 330 may act as storage of fluid evacuated from a respective chamber portion. The extra storage of the optional reservoir 330 allows fluid to evacuate a chamber portion without an active pump. The one or more reservoirs may be high-pressure or low-pressure reservoirs and/or low-volume or high-volume reservoirs for the most effective configuration. For example, one embodiment may have a low-pressure reservoir on the inlet side of the pump, and a high-pressure and high-volume reservoir on the output side of the pump.

The implantable device may include an outer frame (e.g., a stent) constructed of a proper material (e.g., Nitinol wire) with a valve disposed at least partially within. The implantable device may be implanted within a blood vessel such as the superior vena cava, the inferior vena cava, or any other blood vessel where modulating blood flow is desired. The construction of the valve may be of a suitable material such as, a polymer, a biomaterial, a textile, or any other proper material known in the art. The valve may be constructed as a ball disposed in the outer frame and hingedly connected to the outer frame (e.g., a stent). A control element, such as a wire or cable, connected to the ball at a point of leverage with respect to the hinge on the outer frame, may move the ball to the restricted blood flow position when tension is applied by the actuation device.

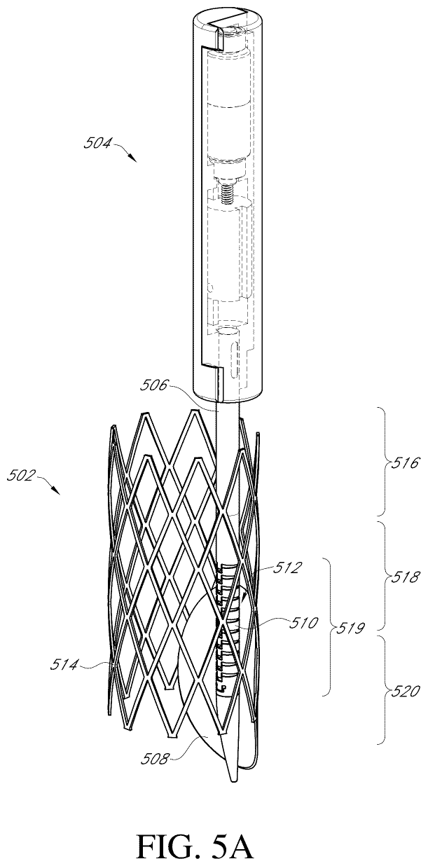

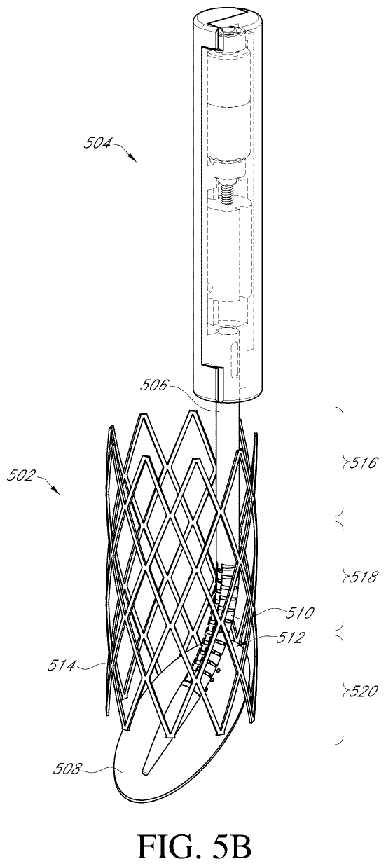

Further, the valve may be a valve flap connected to an articulating arm. This articulating arm, constructed by a plurality of linked segments or by a plurality of laser cut joints with the control element translatable within, is bias to a curved state when tension is applied to the control element via the actuation device (e.g., actuation device 504 shown in FIGS. 5A and 5B). Once in the curved state, the articulating arm holds a valve flap in a closed position. After relaxing tension on the control element, returning the articulating arm back to the unrestricted blood flow position may be done with blood pressure on the valve flap, gravity influences, a relaxed bias of the articulating arm, or combination of any or all. Further, a valve embodiment may be two valve flaps hingedly connected to the outer frame. First and second control elements may couple to first and second portions, respectively, of the valve. The control element may then be coupled to an actuation device and optionally disposed within a sheath. To achieve a restricted blood flow position, the control element is tensioned and retracted within the sheath, drawing the valve flaps closed. Returning the valve flaps to the unrestricted blood flow position may be done by first relaxing tension on the control element, extending it from the sheath, and allowing the valve flaps to reopen due to gravity, blood pressure, or combination of the two.

### Systems and Devices

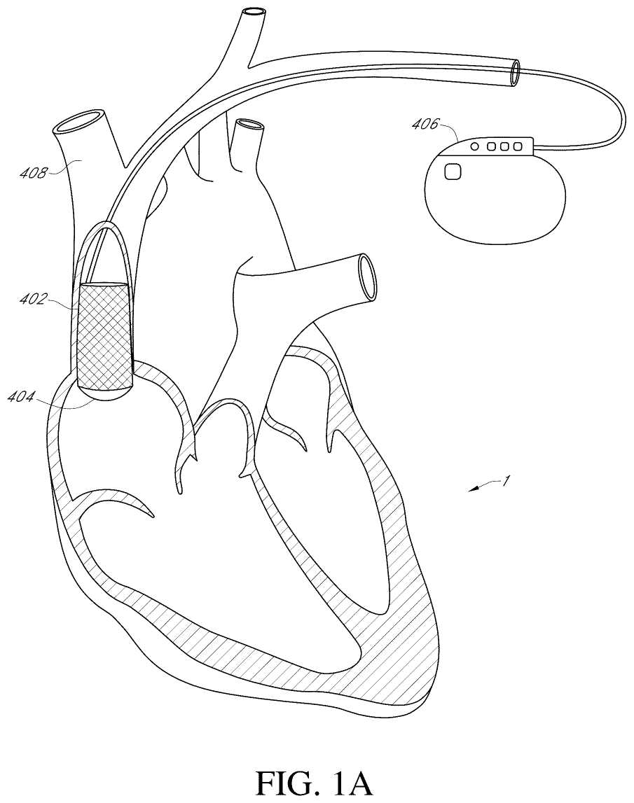

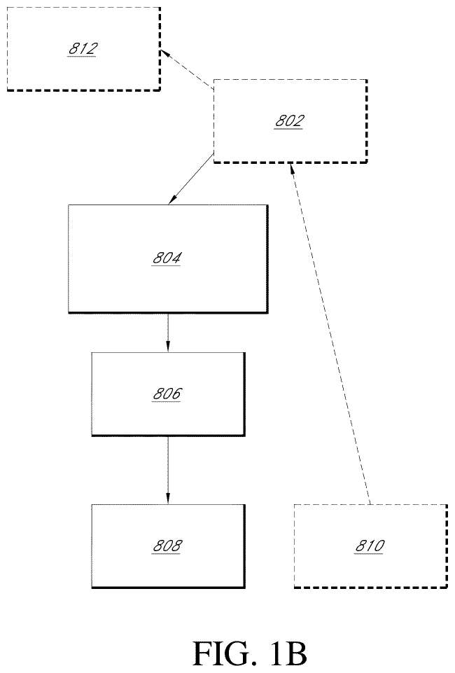

Turning to FIG. 1A, which illustrates an embodiment of a system 1 for modulating blood flow through a blood vessel. A frame 402, such as a stent or similar device, is disposed within a blood vessel 408. The frame 402 at least partially houses a valve which is used to restrict blood flow when certain characteristics (e.g., pressure) are sensed downstream of the valve 404. For example, a sensed characteristic may be a right atrium pressure, such that increased right atrium pressure may result in flow restriction (i.e., decreasing blood flow) in the blood vessel (e.g., SVC, IVC, etc.) being needed, while decreased right atrium pressure may result in flow restriction needing to be alleviated (i.e., returning blood flow to an approximately baseline or original blood flow rate) in the blood vessel. A controller 406 may be responsible for causing activation of an actuation device based on blood flow characteristics sensed by one or more sensors positioned to measure the blood flow characteristics, thus actuating of the valve 404. Although a superior vena cava 408 is shown, it is contemplated herein that the systems and devices can be used in any vessel to regulate flow through the vessel.

FIG. 1B illustrates a block diagram of a system for the modulating of blood flow through a blood vessel. Actuation device 804 is coupled to control element 806, which manipulates leaflet, flap, valve, or valve portion 808. Optionally (shown by dashed lines), controller 802 may be used to control an actuation device 804 to actuate a control element 806 to manipulate a valve 808 between a restricted blood flow position, unrestricted blood flow position, or any position therebetween. In passive control embodiments, as described above, no controller may be present. Controller 802 may include a microprocessor and any other control devices (e.g., operated switches, motor controllers, antennas, or any other common control devices). The controller 802 may optionally receive measurements from one or more optional sensors 810. The one or more sensors 810 may measure properties of the blood it is disposed within (e.g., pressure), a vessel in which the flow modulating device is implanted, or any other physiological or anatomical parameters or properties. Alternatively, or additionally, the controller 802 may be preprogrammed with a rule set or predefined parameters that cause the controller 802 to activate the actuation device based on the rule set and/or predefined parameters. The controller 802 causes the actuation device 804 to adjust the valve 808 to thereby regulate blood flow through a blood vessel. For example, when pressure higher than a pre-defined amount is sensed by a sensor 810 and received by the controller 802, the controller 802 may cause the actuation device 804 and/or control element to move the valve to a restricted blood flow position. Further, when a pressure lower than a pre-defined amount is sensed by a sensor 820 and received by the controller 802, the controller 802 may cause the actuation device 804 to move the valve to an unrestricted blood flow position. Optionally, an antenna disposed in, or within proximity of, the controller 802 may be used to transmit data to a remote computing device 812, for example a server, workstation, physician computing device, or mobile computing device. The transmitted data may include sensor 810 readings, valve position, actuation events, or any other data from the system. Data processing may occur locally on the controller 802 and/or remotely on the remote computing device 812.

FIGS. 2A-2E illustrate example configurations of an actuation device 804 and the corresponding position of the system piston 304, which corresponds to the position of the system valve. In general, actuation device 804 manipulates (e.g., between low pressure states and high-pressure states) a fluid (e.g., liquid, gas, etc.) to move the piston 304 to various positions, corresponding to valve position. These figures are for illustration purposes, and it is disclosed that any aspects or features from FIGS. 2A-2E can be eliminated or combined to achieve desired implementations. In addition, any intermediate positions of the actuation device 804 may be achieved as well and have been contemplated. The illustrated tubing configurations of FIGS. 2A-2E are for illustrative purposes and may not represent literal implementations. The optional input element 324 described below may be a sliding shuttle piston, a stopcock, or any other valving systems known in the art. For example, optional input element 324 may be a sliding shuttle piston constructed with a series of grooved O-rings within a cylinder, such that the tubing configurations of FIGS. 2A-2E may be accomplished by the low-energy act of sliding the shuttle piston to a position that allows communication of respective ports, vents one or more ports, and/or plugs one or more ports. A further example of an optional input element 324 may be a rotational manifold, such as a stopcock valve. The low energy act of rotating the stopcock valve stem may accomplish the tubing configurations of FIGS. 2A-2E by positioning the respective ports in communication with each other, venting one or more ports, and/or plugging one or more ports.

FIG. 2A illustrates an embodiment of an actuation device 804. In this embodiment, the actuation device 804 includes a chamber 307 having a piston 304 movable therein. The chamber 307 includes two portions: a first chamber portion 306 and second chamber portion 308. Actuation device 804 includes a piston 304 with a stroke that is parallel to the piston travel line 302. Further, a first port 310 and a second port 312 are fluidly connected to a manifold 318. Additionally, an inlet port 314 (e.g., suction) and an output port 316 (e.g., pressure) are fluidly connected to the manifold 318. The manifold 318, using an optional input element 324 in some embodiments, may be manipulated to cause fluid communication of one or more ports of the actuation device 804 for different positions of the piston 304. The manipulation of the manifold 318 between configurations may be from a self-contained device or an external control device.

In an example position, as depicted in FIG. 2A, the first port 310 is fluidly connected to the inlet port 314 (e.g., suction), and the second chamber portion 308 is fluidly connected to the output port 316 (e.g., pressure). As depicted, the inlet port 314 (e.g., suction) is fluidly connected to an inlet 327 of a pump 322, in some embodiments, through a one-way valve 328 oriented with flow to the pump 322. The pump output 325 is fluidly connected to a reservoir 320, enabling the reservoir 320 to be charged (e.g., pressurized) by the pump 322. Some embodiments include a one-way valve 326 from the pump 322 to the reservoir 320. One or both one-way valves 328 and 326 may be check valves (e.g., ball and seat check valves). When the pump 322 is active, the depicted plumbing creates suction at the inlet port 314, which leads to inlet 327 of the pump 322. The depicted plumbing further creates fluid pressure in the reservoir 320 from the pump 322, when the pump 322 is active. Additionally, the depicted plumbing creates pressure from the reservoir 320 out the output port 316 when the pump is active or when fluid pressure is stored. The depicted plumbing configuration evacuates fluid from the first chamber portion 306 and injects fluid into the second chamber portion 308, thus causing the piston 304 to move toward and/or into the first chamber portion 306. This piston 304 movement may cause slack or release tension in the control element of the valve, positioning the valve in the unrestricted blood flow position.

FIG. 2B depicts the actuation device 804 with the manifold 318 reconfigured to connect the second port 312 with the inlet port (e.g., suction) 314 and connect the first port 310 with the output port (e.g., pressure) 316. In the configuration of FIG. 2B, the actuation device 804 evacuates fluid from the second chamber portion 308 and injects fluid into the first chamber portion 306, thus causing the piston 304 to move toward and/or into the second chamber portion 308. This piston 304 movement can tension the control element of the valve, thereby positioning the valve in the restricted blood flow position. Additionally shown in FIG. 2B is an optional reservoir 330. The optional reservoir 330 may provide temporary storage of fluid coming from the manifold 318 or chamber 307 and ultimately into the pump 322, reducing vacuum effects on the inlet tubing. This optional reservoir 330 may be present on any of the actuation device embodiments described herein, for example any of FIGS. 2A-2E.

In an example position, as depicted in FIG. 2C, the first port 310 is fluidly connected to the inlet port 314 (e.g., suction) through a reservoir 330. Further, the second chamber port 308 is fluidly connected to the output port 316 (e.g., pressure). As depicted, the inlet port 314 (e.g., suction) is fluidly connected to an inlet 327 of a pump 322, in some embodiments, through a one-way valve 328 oriented with flow to the pump 322. A reservoir 330 is included in the plumbing leading to the inlet port 314. Benefits of a reservoir 330 on the input side of the pump may include auxiliary fluid volume to avoid vacuum effects in the intake tubing. Additionally, the extra fluid storage of the reservoir 330 allows fluid to be evacuated from a respective chamber portion without an active pump 322. In other words, embodiments without the reservoir 330 are unable to evacuate fluid or are limited in the amount of fluid that can be evacuated without an active pump 322. In said embodiments, equalization of the pressure on the evacuation lines and the reservoir 320 may be created, thus ceasing actuation of the piston 304 until actuation of the pump 322. The output port 316 (e.g., pressure) is fluidly connected to a reservoir 320, in some embodiments, enabling the reservoir 320 to be charged by the pump 322. Some embodiments include a one-way valve 326 from the pump 322 to the reservoir 320. Both the one-way valves 328 and 326, may be check valves (e.g., ball and seat check valves). The depicted plumbing creates suction at the inlet port 314, which leads to inlet 327 of the pump 322, when the pump 322 is active. The depicted plumbing further creates fluid pressure in the reservoir 320 from the pump 322, when the pump 322 is active. Additionally, the depicted plumbing creates pressure from the reservoir 320 out the output port 316 when the pump is active or when fluid pressure is stored. The depicted plumbing configuration evacuates fluid from the first chamber portion 306 and injects fluid into the second chamber portion 308, thus causing the piston 304 to move toward and/or into the first chamber portion 306. This piston 304 movement may cause slack or release tension in the control element of the valve, positioning the valve in the unrestricted position.

FIG. 2D depicts another embodiment of the actuation device 804. This embodiment does not have a second port in the second portion 308 of the chamber 307 but, optionally, includes a compression spring 332 in the chamber 307. The compression spring 332 may be biased to return the piston 304 to the unrestricted blood flow position, as shown. When the first port 310 is fluidly connected to the inlet 327 of the pump 322, the compression spring 332 aids in returning the piston 304 to the unrestricted blood flow position. FIG. 2E illustrates the inverse of this as the first port 310 of chamber 307 is fluidly connected to the output port 316 (e.g., pressure). The injection of fluid into the first chamber portion 306 of chamber 307 drives the piston 304 towards the compression spring 332. This piston 304 action compresses the compression spring 332, creating the force necessary to return the piston 304 to the unrestricted position when injection pressure is removed (e.g., as shown in FIG. 2D). The embodiments of FIGS. 2D-2E may use a pumping pressure and a piston 304 cross-sectional area large enough to generate a force large enough to overcome the compression force of the compression spring 332 in the restricted blood flow configuration. Alternatively, the compression spring 332 could be located in the first chamber portion 306, the second port 312 could be used, and the first port 310 could be removed. In this described embodiment, fluid could be injected into the second chamber portion 308 to move the piston 304 into the unrestricted position. Inversely, fluid could be evacuated from the second chamber portion 308, aided by the compression spring, to move the piston 304 into the restricted position.

Further embodiments may use tension springs located inversely to the compression springs described above. These tension springs may require proper attachment but could generate the same forces as the compression springs 332 described above. For example, the illustrated embodiment of FIGS. 2D-2E could instead have a tension spring within the first chamber portion 306. When fluid is injected into the first chamber portion 306 to move the piston 304 to a restricted blood flow position, the tension spring could store the energy necessary to pull the piston 304 back to the unrestricted position when fluid is evacuated from the first chamber portion. Further contemplated are embodiments that could be configured the same as the embodiments described above but without springs. These embodiments would use injection pressure from the output port 316 (e.g., pressure) into the first port 310 to move the piston 304 into the restricted position or injection pressure from the output port 316 into the second port 312 to move the piston 304 into the unrestricted blood flow position. Inversely, suction pressure from the suction port 314 into the first port 310 may be used to move the piston 304 into the unrestricted position or suction pressure from the suction port 314 into the second port 312 may be used to move the piston 304 into the restricted blood flow position.

In the embodiments of FIGS. 2A-2E, a third configuration of the manifold 318 is further contemplated. In this configuration, the manifold 318 isolates the first port 310 and second port 312 (in embodiments with one or the other, or both). Further, the inlet port 314 (e.g., suction) and output port 316 (e.g., pressure) may be isolated or fluidly connected. In embodiments with a continuous pump (e.g., anatomical pump), it may be advantageous to fluidly connect the inlet port 314 to the output port 316, so that the pump 322 can recirculate and not build excessive pressure. In embodiments with an intermittent pump 322 (e.g., electromechanical pump), the inlet port 314 and second port 312 (e.g., output port) may be isolated. By doing so and isolating the first port 310 and second port 312, the piston 304 can be stopped and held in any intermediate position, at or between, unrestricted and restricted blood flow positions.

Further, embodiments of the actuation device 804 without the pump 322 and reservoir 320 have been contemplated. These embodiments may utilize the anatomical pressures (e.g., blood pressure) for the actuation of the piston 304. Further, these embodiments may have access to a high-pressure point of the anatomy fluidly connected to the output port 316 and access to a low-pressure point of the anatomy fluidly connected to the inlet port 314. Configured in the anatomy in this way, these embodiments may achieve the actuation capabilities of any of the preceding embodiments. Depending on the manifold 318 configuration, fluid pressure from the anatomy may be injected from the output port 316 into either the first chamber portion 306 or the second chamber portion 308 or vented into the inlet port 314 from either the first chamber portion 306 or the second chamber portion 308. In further embodiments, the reservoir 320 may be filled or pressurized extracorporeally by the patient or a healthcare professional on a periodic or on as-needed basis. Access tubing to the reservoir may be routed, with appropriate valving, for subcutaneous access.

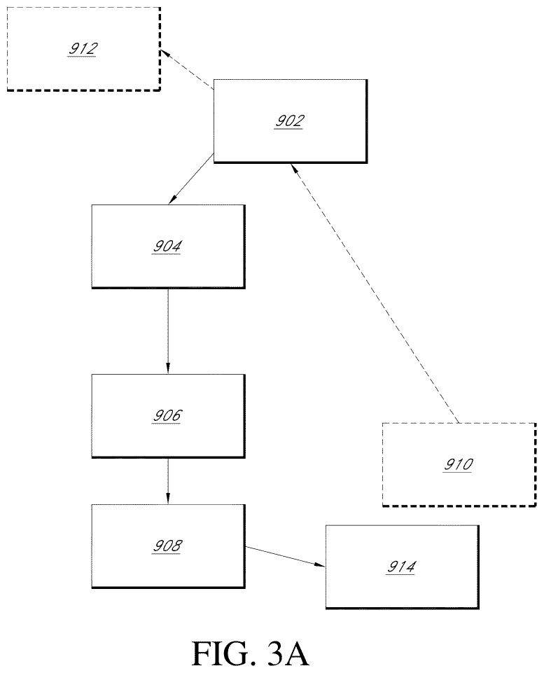

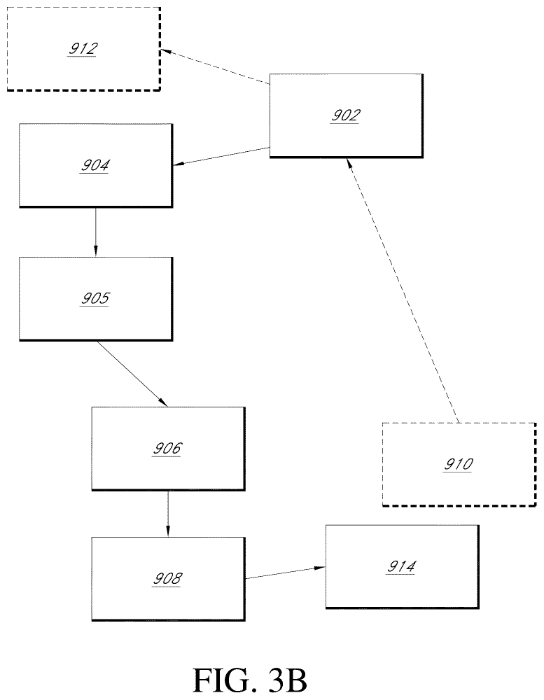

FIG. 3A depicts another embodiment of a system for modulating blood flow through a blood vessel. A system for modulating blood flow may include a first magnet 906, an actuation device 908, and a valve 914. The first magnet 906 is operatively coupled to the actuation device 908, and the actuation device 908 is operatively coupled to the valve 914 to effect movement of the valve 914. The system may include a control device 902 operatively coupled to a second magnet 904. The control device 902 can include a microprocessor, power source (a battery, a capacitor, wall outlet, or any other suitable power source), antenna, operated switches, and/or any other control devices. The control device 902 and second magnet 904 may be located externally but proximal to a user. Alternatively, the control device 902 and second magnet 904 may be implanted (e.g., subcutaneously, intravascularly, etc.). Optionally, a system for modulating blood flow may include a sensor 910. The sensor 910 may sense one or more physiological or anatomical attributes and output a signal to the control device 902, which may output an activation signal to the actuation device 908 to tension or release tension in the control element.

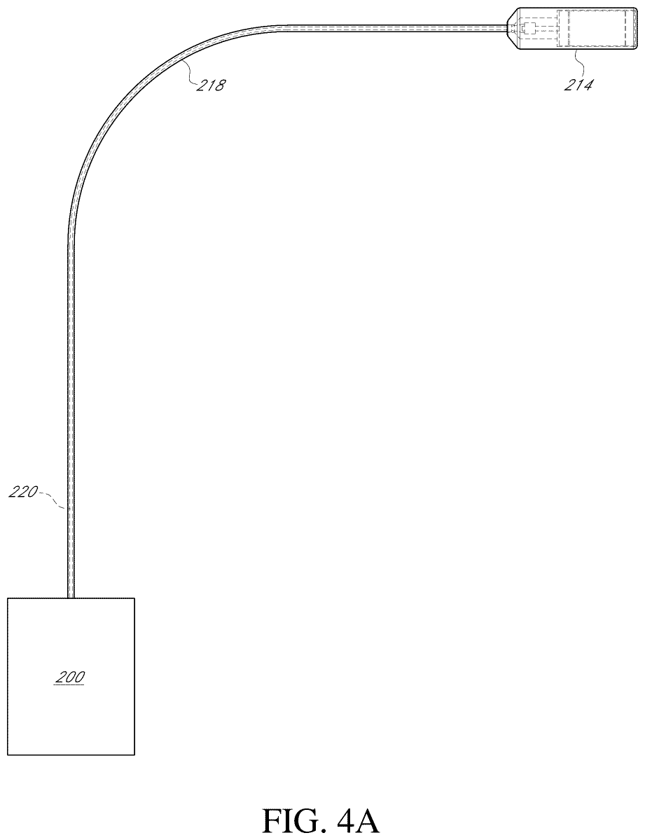

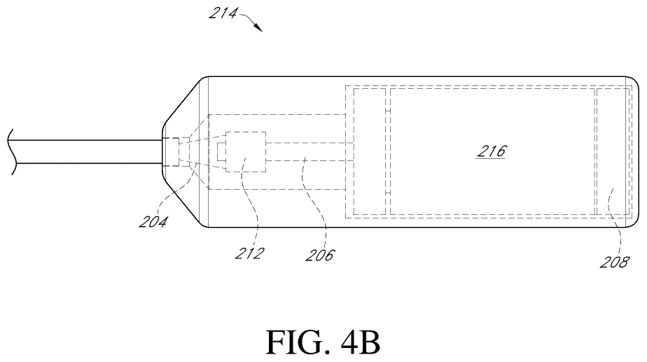

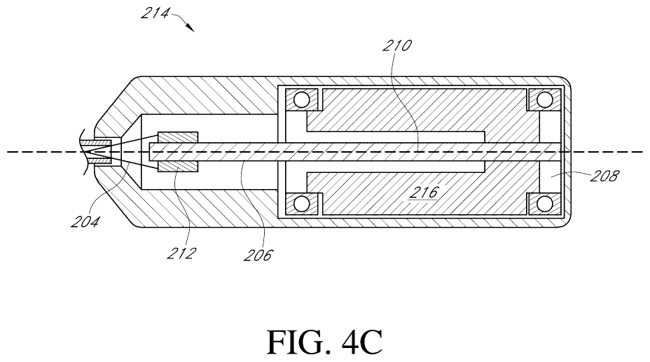

The actuation device 908 may be a linear actuator coupled to the control element of the valve 914. The second magnet 904, although external to the user or implanted at a second location (the implantable device being at a first location), is placed operationally proximal to the first magnet 906. By doing so, the magnetic pole orientation of the second magnet 904 influences the magnetic pole direction of the first magnet 906. In other words, a magnetic gear train may be created between the second magnet 904 and the first magnet 906, such that when the control device 902 rotates the second magnet 904, the first magnet 906 is rotated in an opposing direction (much like two mated gears). Rotating the first magnet 906 induces movement in the actuation device 908, which tensions or releases tension in the control element, which moves the valve 914 to a restricted or unrestricted blood flow position, respectively. In embodiments, shown in FIGS. 4A-4C, with an actuation device 908 being a linear actuator connected to the control element, the rotation of the first magnet 906 actuates the linear actuator (e.g., lead screw) to slack or tension the control element. In some embodiments, both the first magnet 906 and the second magnet 904 may be permanent magnets. In further embodiments, the first magnet 906 is a permanent magnet and the second magnet 904 is an electromagnet. As such, the second magnet 904 may not be rotated to influence the position of the first magnet 906. With the angular momentum of the first magnet 906 (after being made to move) and alternating the polarity of the second magnet 904 by alternating the direction of current in the coils of the second magnet 904 in a predetermined pattern.

As also described elsewhere herein, the control device 902 may receive signals from one or more optional sensors 910. These signals may be indicative of characteristics of total blood volume in the vasculature. For example, when the control device 902 receives a signal indicative of a measured pressure higher than a pre-defined amount, the control device 902 can cause the second magnet 904 to rotate. The rotation of the second magnet 904 causes the first magnet 906 to rotate, which actuates the actuation device 908 to move the valve 914 towards a restricted blood flow position. Further, when the control device 902 receives a signal indicative of a measured pressure lower than a pre-defined amount, the control device 902 may cause the second magnet 904 to rotate in an opposing direction. The rotation of the second magnet 904 causes the first magnet 906 to rotate, thereby actuating the actuation device 908 to move the valve 914 towards the unrestricted blood flow position. Sensor signals from sensor 910 may be transmitted to control device 902 via a wired connection or wirelessly. The described system is capable of positioning the valve in any position on or between the restricted position and unrestricted position. Additionally, and optionally, the system may include the capability of transmitting data to a remote computing device 912. The transmitted data may include sensor 910 measurements, valve position, actuation events, or any other data from the system.

FIG. 3B is an embodiment of a system for modulating blood flow through a blood vessel. Similar to FIG. 3A, FIG. 3B shows a magnetic gear train for manipulation of a valve of an implanted device. The control device 902 and second magnet 904, as in FIG. 3A, may be located external to the user or implanted (e.g., subcutaneously, intravascularly, etc.). Similar to FIG. 3A, the first magnet 906, actuation device 908, valve 914, and optional sensor 910 may be implanted within the user. Unlike FIG. 3A, this embodiment uses an implanted (in some embodiments, implanted subcutaneously) repeater magnet 905. This repeater magnet 905 may be used to extend the operational distance between the second magnet 904 and the first magnet 906, as it is implanted at an appropriate position between the two. Additionally, the repeater magnet 905 may be used to increase the torsional force that can be applied by the magnetic gear train. Further contemplated embodiments include a repeater module with a second power source operatively coupled to the repeater magnet 905 and capable of powering the rotation of the repeater magnet 905. Further, the rotation direction of the second magnet 904 and first magnet 906 are now the same, not opposing one another as in the previous embodiment. When manipulating the valve 914 towards the restricted or unrestricted blood flow positions, the second magnet 904 can be rotated in the same direction as the desired direction of the first magnet 906.

FIG. 4A depicts a system for modulating blood flow through a blood vessel. As depicted, an implantable device 200, including an outer frame, is implantable within a blood vessel. A valve of implantable device 200 is actuated by a control element 220 (e.g., a wire or cable), which is disposed within a sheath 218 coupled to an actuation device 214. Actuation device 214 may be similar to the embodiments described in connection with FIGS. 3A-3B. Shown in a zoomed-in view in FIG. 4B, the actuation device 214 may include a magnet 216 coupled to a lead screw 206, which is operatively coupled to nut 212, which is operatively coupled to a control element 204. As in FIGS. 3A-3B, when the magnet 216 (first magnet 906 in FIGS. 3A-3B) is driven to rotate, the nut 212 moves along lead screw 206 to extend or retract control element 204. When retracted (moves on lead screw 206 towards magnet 216), the nut 212 tensions the control element 204, which moves the valve to a restricted blood flow position. When extended (moves on lead screw 206 away from magnet 216), the nut 212 relieves tension on the control element 204, which moves the valve to an unrestricted blood flow position. FIG. 4C depicts how the magnet 216 and lead screw 206 can be made to rotate about the longitudinal axis 210, which causes the nut 212 to translate along the longitudinal axis 210. When rotating the lead screw 206 within the nut 212, frictional forces on the thread interface between the nut 212 and the lead screw 206 may cause the nut 212 to rotate with the lead screw 206. Some embodiments include a keyed nut 212 mated with a key inserted into or formed from the housing of the actuation device 214. Wherein the nut 212 can translate within the key. Other embodiments may use a sleeve formed from or inserted into the housing of the actuation device 214. Wherein the sleeve matches the geometry of the nut 212, which is any geometric shape not suitable for rotation, and in which the nut 212 can translate within. In alternative embodiments that do not employ a lead screw and a nut, a spool like element may be employed such that rotation (via the magnet) of the spool in a first direction causes the control element 204 to wrap around the spool to actuate the valve to a restricted blood flow position. Rotation of the spool in a second direction, opposite the first direction, causes the spool to release the control element 204 to relax the valve to an unrestricted blood flow position.

FIG. 4A includes an embodiment in which the actuation device 214 includes a microprocessor, power source, and an electromechanical linear actuator. The actuation device 214 can actuate the nut back and forth by supplying power to the linear actuator. The control device may have the capability (e.g., H-bridge motor controller) to supply a voltage with a first current direction to the linear actuator and a voltage with a second current direction, opposite the first direction, to cause the linear actuator motor to rotate in either direction for advancing and/or retracting the nut. Further contemplated are actuation devices 214 with hydraulic or pneumatic linear actuators. These linear actuators act much like piston-based hydraulic cylinders. When compressed gas or liquid is injected into the piston chamber, the piston moves, pulling on the control element. Once the compressed gas or liquid is vented from the piston chamber, the piston returns and relieves tension on the control element. A microprocessor of the system may receive measurements from one or more sensors, and when the measurement is higher than a pre-defined threshold, the actuation device can move the valve toward a restricted blood flow position. Alternatively, when the measurement is lower than a pre-defined threshold, the actuation device can move the valve toward an unrestricted blood flow position. These embodiments are capable of positioning the valve in any position at, or between, the restricted blood flow position and unrestricted blood flow position.

FIGS. 5A and 5B illustrate an implantable device 502 including an articulating arm 519 that manipulates the valve 508. Any actuation device described herein may be used in the place of actuation device 504. The control element, as shown in FIGS. 5A-5B, includes an articulating arm 519 including a tube 506 and linkage mechanism 510. The articulating arm 519 is articulated via a pullwire or cable disposed within it, such that the actuation device 504 tensions or slacks the pullwire or cable to cause articulation or relaxation of the articulating arm 519. The linkage mechanism 510 may be constructed of a tube with a plurality of laser cut joints or constructed with a series of linked segments. When tension is applied to the pullwire within the articulating arm 519, the linkage mechanism 510 is biased to a curved state, as shown in FIG. 5B. When tension is released on the pullwire within the articulating arm 519, the linkage mechanism 510, either biased toward or assisted by blood pressure, is moved to a substantially linear position or uncurved position or relaxed configuration, as shown in FIG. 5A. Valve 508 is coupled (e.g., hingedly connected) to the outer frame 514, for example at a distal end 520 of outer frame 514 at an intermediate position 518 on the outer frame 514, or a proximal end 516 of outer frame 514. Valve 508 is pivoted to the restricted blood flow position, as shown in FIG. 5B, when tension is applied to the pullwire in the articulating arm 519. The valve 508 returns to the unrestricted blood flow position, as shown in FIG. 5A, when tension is released on the pullwire in the articulating arm 519. The valve 508 of implantable device 502 of FIGS. 5A and 5B can be actuated to and held at any position between and including the restricted blood flow position and unrestricted blood flow position. Although a pullwire is described as the mechanism that articulates articulating arm 519, it will be appreciated that any articulating mechanism may be used such as: concentric tubes, where the tubes are selectively bonded together such that manipulation of the inner tube deflects the outer tube; robotically controlled joints; and the like.

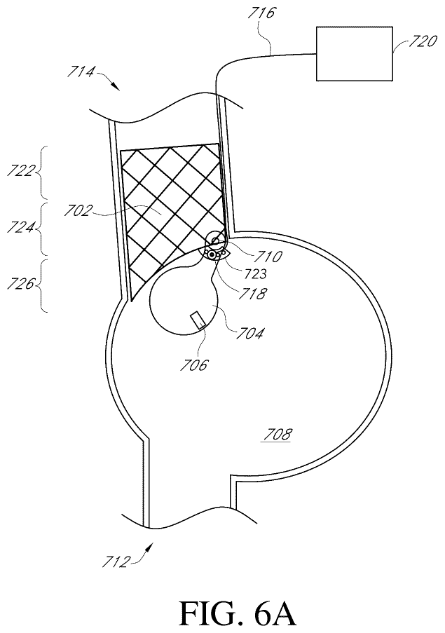

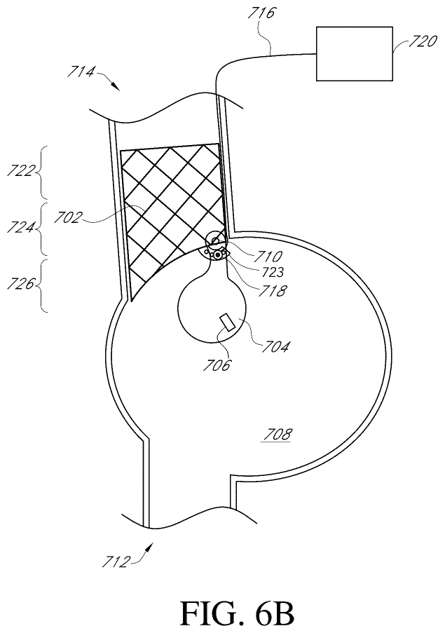

FIGS. 6A and 6B illustrate another embodiment of an implantable device for modulating blood flow through a vessel 714. The valve of this embodiment includes a ball 704 hingedly coupled to an outer frame 702. The ball 704 may be hingedly coupled to a distal end 726 of the outer frame 702, an intermediate position 724 of the outer frame 702, or a proximal end 722 of the outer frame 702. The ball 704 can be actuated from an unrestricted blood flow position, as shown in FIG. 6B, to a restricted blood flow position, as shown in FIG. 6A, by the tensioning of a control element 716. Returning the ball 704 to an unrestricted blood flow position may be done by relaxing tension on the control element 716 and allowing the ball 704 to drop back to the unrestricted blood flow position due to gravity and/or blood pressure. Any actuation device described herein may be used as actuation device 720. Additionally, shown in FIGS. 6A-6B is exemplary anatomy that the device may be disposed within. The outer frame 702 is depicted as being in the super vena cava, the ball 704 is depicted in the right atrium 708, with the right atrium 708 having the inferior vena cava 712 as an outlet.

In some embodiments, the ball 704 may be locked in position by a solenoid lock 718. The solenoid lock 718 may be a latching solenoid. Latching solenoids may be more efficient than traditional solenoids as they only require a pulse of electric current to move from an unlatched state to a latched state and only a reverse polarity current pulse to move from a latched state to an unlatched state. A traditional solenoid may require continuous current to hold a deployed position.

In some implementations, the outer frame 702 may include a wing portion 723 with material removed to define one or more apertures sized for the pin of the locking solenoid 718. The alignment of the one or more defined apertures with the pin of the locking solenoid 718 may correspond to one or more desired positions of the ball 704. For example, when the ball 704 is actuated to the restricted blood flow position by tensioning the control element 716, the solenoid lock 718 may be actuated to slide a pin into the corresponding aperture. Once the ball 704 is to return to the unrestricted blood flow position, the solenoid lock 718 may be then actuated to pull the pin from the aperture before, while, or after the control element 716 is relaxed.

Additionally, and optionally, one or more sensors 706 may be disposed on the ball 704. The one or more sensors 706 may measure physical properties of the blood flow (e.g., pressure) it is disposed in. The one or more sensor measurements may be received by a control device (e.g., controller 802 shown in FIG. 1B), which causes the control device to output signals to an actuation device 720, which may change the ball 704 position.

Sensors described herein may be of a variety including: strain gauges, piezoelectric sensors, capacitance sensors, vacuum sensors, and any other variety sensor appropriate and known in the art. Further, sensors described herein may be used in the right atrium of the heart of a user and may be used to sense an increase or decrease of pressure. Further, sensors described herein may installed anywhere appropriate for desired measurements, including: the outer frame, the valve, the control device, the actuation device, and the like. Further, one or more sensors may be used in conjunction with any of the devices and systems herein to measure one or more physical characteristics of the patient. For example, it may be beneficial to measure whether the patient is standing, sitting, or laying. In addition, the pressure thresholds for activating the device may be influenced by the activity of the patient. For example, it may be beneficial to recognize that the patient is exercising, as this would elevate pressures and may cause necessitate adjustments in pressure thresholds. Characteristics described above may be measured by a pressure sensor in blood vessels of other portions of the body, a gyroscopic sensor for changes in angular position, an accelerometer for changes in acceleration (e.g., orientation recognition with regard to the direction of gravity), a heart rate sensor, a sensor measuring a size of a blood vessel, or any other sensors for measuring physical characteristics. The described characteristics, individually or in combination, may be received by a microprocessor and processed to effect changes in valve position (e.g., using an actuating device) based on the sensed characteristics.

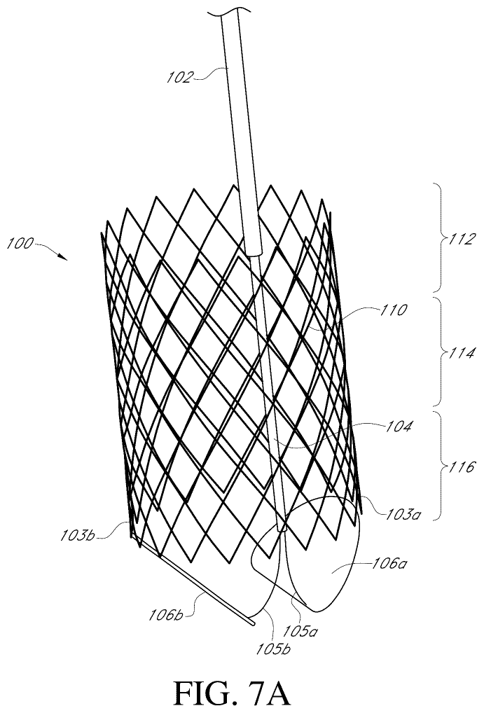

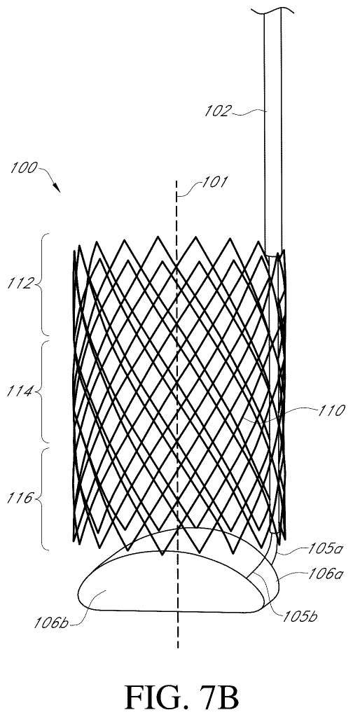

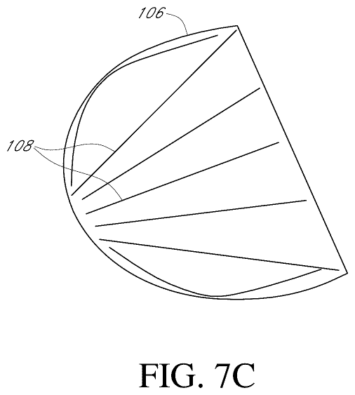

FIGS. 7A-7B depict an embodiment of an implantable device 100 for modulating blood flow through a vessel. As shown in FIG. 7A, the valve includes a first valve portion 106a and a second valve portion 106b. The control elements 105a, 105b (e.g., a wire or cable) may include first and second control elements 105a, 105b, which each attach to a respective valve portions 106a, 106b. Optionally, the first and second valve portions 106, 106b can be hingedly connected to the outer frame at valve attachment portions 103a, 103b. First valve attachment portion 103a may be positioned on first valve portion 106a at a location that is opposite where the first valve portion 106a meets (e.g., substantially at a central axis 101, shown in FIG. 7B, of the implantable device 100) the second valve portion 106b. Similarly, the second valve attachment portion 103b may be positioned on second valve portion 106b at a location that is opposite of where the second valve portion 106b meets (e.g., substantially at a central axis 101 of the implantable device 100) the first valve portion 106a. When moving this valve embodiment to a restricted blood flow position, the control elements 105a, 105b are tensioned and the tension pulls the valve portions 106a, 106b close together. The valve portions 106a, 106b may be hingedly attached or be constructed of a material flexible enough to articulate from an unrestricted blood flow position to a restricted blood flow position. Additionally, it may be effective to attach the valve portions 106a, 106b to a distal end 116 of the outer frame 110, an intermediate position 114 (i.e., a position between the distal end 116 and the proximal end 112) of the outer frame 110, or a proximal end 112 of the outer frame 110. It may be effective to use a sheath 104 disposed in a catheter 102 to contain at least a portion of the control elements 105a, 105b. The sheath 104 and/or catheter 102 may be positioned in such a way to limit the stroke of the control elements 105a, 105b. In other words, the valve portions 106a, 106b may contact a distal face of the sheath 104 and/or catheter 102 when the valve reaches the restricted blood flow position. The valve may be returned to the unrestricted blood flow position by relaxing the tension on the control elements 105a, 105b, allowing the control elements 105a, 105b to re-extend from the sheath 104. Blood pressure and/or gravity may create the force that returns the valve portions 106a, 106b of the implantable device 100 back to the unrestricted blood flow position. Additionally, the valve portions 106a, 106b may be constructed of a material with shape memory qualities and that is flexible enough to articulate from an unrestricted blood flow position to a restricted blood flow position. As such, the valve portions 106a, 106b may be coupled to the outer frame 110 in the unrestricted blood flow position, generating forces for returning the valve portions 106a, 106b to the unrestricted blood flow position when held in the restricted blood flow position. This valve embodiment may be actuated to and held at any position including, or between, the restricted position and the unrestricted blood flow position.

FIG. 7C depicts an embodiment of a valve or valve portion. The valve or valve portions (e.g., 106a or 106b) may have reinforcing veins 108. These veins may be of appropriate materials to supply increased rigidity, increased tensional strength, and/or any other desired physical attributes to the valve or valve portions 106a, 106b. Vein 108 materials may include fibers, injected epoxies, polymers, alloy strands (e.g., Nitinol), or any other appropriate material known in the art.

### Methods

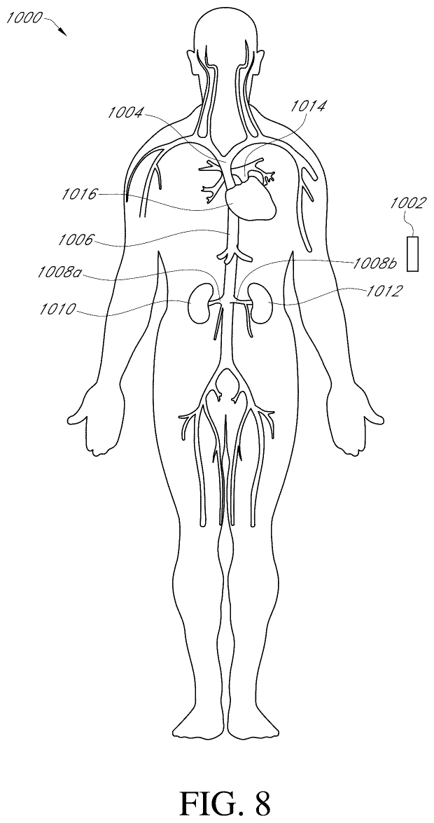

FIG. 8 illustrates a schematic representation of portions of a subject 1000. The flow modulating devices described herein (e.g., represented in FIGS. 1A-1B, 3A-3B, 4A, 5A-7B) may be introduced (e.g., implanted) in vasculature of the body. In general, the device 1002 may represent any of the flow modulating devices described herein (e.g., shown in FIGS. 1A-1B, 3A-3B, 4A, 5A-7B) and may include the same or similar functionality and/or structures. In some examples, the device 1002 may be implanted in or near to a portion of the Superior Vena Cava (SVC) 1004. In some examples, the device 1002 may be implanted in or near to a portion of the Inferior Vena Cava (IVC) 1006. The subject 1000 is illustrated with a representation of a portion of the vasculature system to generally illustrate the SVC 1004 and the IVC 1006 within the subject 1000. However, it is to be understood that no dimensions or relative sizes of components may be inferred from the relative sizes and dimensions of elements in the figures.

The subject 1000 includes a number of vessels and organs that may circulate blood throughout the body. For example, renal veins 1008a and 1008b drain blood from respective right kidney 1010 and left kidney 1012. Renal veins 1008a and 1008b connect to the IVC 1006. Blood from the aorta 1014 flows to the IVC 1006. Blood travels from the aorta 1014 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1006 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure patients can have multiple comorbidities that cause excessive congestion or accumulation of blood volume in the splanchnic venous circulation. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. These issues can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized.

For example, because blood flow from the splanchnic venous circulation is directed through hepatic veins and into the IVC 1006, devices (as described herein) may be placed into the IVC 1006 to limit blood flow to allow the splanchnic venous circulation to expand with increased blood volume. Placing devices (as described herein) into the IVC 1006 may also allow the body to accumulate blood volume in the splanchnic venous circulation, which can maximize the downstream drop of pressure relative to upstream increase of pressure. Similarly, devices (as described herein) may be placed into the SVC 1008 to limit blood flow to allow the reservoir to expand with increased blood volume. Furthermore, the flow modulating devices described herein may be placed in either the IVC 1006 and/or SVC 1008 to alleviate pressure in the right side of the atrium of the heart 1016 and/or regulate renal venous pressure and kidney function. Another position example of a flow modulating device may be in the IVC below the renal veins. Positioning the flow modulating device in the IVC below the renal veins may have a similar effect as the SVC location, as it may allow the flow modulating device to maintain renal venous pressure, which can correlate with sustained renal function and diuresis.

In some examples, the flow modulating device 1002 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1002 may be used as a method of treatment to regulate pressure in the right atrium of the heart. Further, the flow modulating device 1002 may be used as a method of treatment to improve function of the kidneys in patients having reduced kidney function due to pressure in the venous system.

For example, any of the implantable devices and/or systems described herein may be configured to modulate a volume of blood flowing from a superior vena cava into a right atrium to decrease right atrial pressure.

Further for example, any of the implantable devices and/or systems described herein may be used to perform a method including restricting blood flow within a blood vessel.

Still further for example, any of the implantable devices and/or systems described herein may be used to perform a method of treatment for a subject having one or both of: congestive heart failure or chronic kidney disease. The method may include restricting blood flow within the blood vessel.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the actuating device, the controller device, and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

### EXAMPLE EMBODIMENTS

Example 1. A system for modulating blood flow through a blood vessel, the system comprising: an implantable device comprising: an outer frame, a valve disposed in the outer frame, and a control element coupled to the valve, wherein the control element is configured to manipulate the valve between an unrestricted blood flow state and a restricted blood flow state; and an actuation device comprising: a pump fluidly connected to a reservoir, a chamber comprising a first portion and a second portion, a manifold fluidly connected to the pump, the reservoir, and the chamber, wherein the manifold comprises at least one port configured to fluidly connect the reservoir to the first portion of the chamber, and a piston coupled to the control element and configured to move between a restricted blood flow position and an unrestricted blood flow position within the chamber, wherein: the piston is configured to move to the restricted blood flow position when a fluid is flowed from the reservoir through the manifold into the first portion of the chamber, and the piston is configured to return to the unrestricted blood flow position when the fluid is evacuated from the first portion of the chamber.

Example 2. The system of any the preceding examples, but particularly Example 1, wherein the fluid comprises a gas.

Example 3. The system of any the preceding examples, but particularly Example 1, wherein the fluid comprises a liquid.

Example 4. The system of any the preceding examples, but particularly Example 1, wherein the fluid in the reservoir is a rechargeable fluid.

Example 5. The system of any the preceding examples, but particularly Example 1, wherein the actuation device further comprises a power source.

Example 6. The system of any the preceding examples, but particularly Example 5, wherein the pump comprises an electromechanical pump such that the power source is configured to electrically power the pump.

Example 7. The system of any the preceding examples, but particularly Example 1, wherein the pump is configured to be activated by an anatomy of a user.

Example 8. The system of any the preceding examples, but particularly Example 1, wherein the manifold comprises a second port fluidly connected to the second portion of the chamber, wherein the piston is configured to move to the unrestricted blood flow position when the fluid enters the second port from the reservoir via the manifold, thereby causing the valve of the implantable device to move to the unrestricted blood flow state.

Example 9. The system of any the preceding examples, but particularly Example 8, wherein the piston is further configured to move to an intermediate position, wherein the fluid evacuates the second portion of the chamber through the second port into the reservoir through the manifold.

Example 10. The system of any the preceding examples, but particularly Example 1, wherein the manifold comprises a second port fluidly connected to a second portion of the chamber, wherein the fluid is configured to be evacuated from the second portion of the chamber through the second port when the piston is in the restricted blood flow position.

Example 11. The system of any the preceding examples, but particularly Example 10, wherein the manifold further comprises an input element configured to direct flow from one of: the reservoir to the first portion of the chamber, the first portion of the chamber to the pump, the reservoir to the second portion of the chamber, or the second portion of the chamber to the pump.

Example 12. The system of any the preceding examples, but particularly Example 10, further comprising a second reservoir, wherein the fluid is configured to flow from the second portion of the chamber to the second reservoir.

Example 13. The system of any the preceding examples, but particularly Example 12, wherein the fluid is configured to be pumped from the second reservoir to the reservoir.

Example 14. The system of any the preceding examples, but particularly Example 12, wherein the second reservoir comprises a low-pressure reservoir.

Example 15. The system of any the preceding examples, but particularly Example 1, wherein the fluid connection between the pump and the reservoir comprises a one-way valve to enable fluid flow from the pump to the reservoir.

Example 16. The system of any the preceding examples, but particularly Example 15, wherein the one-way valve comprises a check valve.

Example 17. The system of any the preceding examples, but particularly Example 1, wherein the fluid connection between the manifold and the pump comprises a one-way valve to enable the fluid to flow from the manifold to the pump.

Example 18. The system of any the preceding examples, but particularly Example 17, wherein the one-way valve comprises a check valve.

Example 19. The system of any the preceding examples, but particularly Example 1, wherein the at least one port further fluidly connects the first portion of the chamber to the pump through the manifold.

Example 20. The system of any the preceding examples, but particularly Example 19, wherein the at least one port is fluidly connected to the pump through the manifold to evacuate the fluid from the first portion of the chamber thereby moving the piston to the unrestricted blood flow position.

Example 21. The system of any the preceding examples, but particularly Example 19, wherein the piston comprises a spring piston, wherein the spring piston is configured to automatically return to the unrestricted blood flow position when the fluid is evacuated from the first portion of the chamber.

Example 22. The system of any the preceding examples, but particularly Example 1, wherein the reservoir is a high-pressure reservoir.

Example 23. The system of any the preceding examples, but particularly Example 1, wherein the reservoir is a high-volume reservoir.

Example 24. The system of any the preceding examples, but particularly Example 1, wherein the outer frame of the implantable device comprises a stent.

Example 25. The system of any the preceding examples, but particularly Example 1, wherein the implantable device further comprises an articulating arm, wherein the control element is disposed in the articulating arm.

Example 26. The system of any the preceding examples, but particularly Example 25, wherein the control element is configured to actuate the articulating arm, thereby actuating the valve between the unrestricted blood flow state and the restricted blood flow state.

Example 27. The system of any the preceding examples, but particularly Example 25, wherein the articulating arm comprises a plurality of laser cut joints.

Example 28. The system of any the preceding examples, but particularly Example 25, wherein the articulating arm comprises a plurality of linked segments.

Example 29. The system of any the preceding examples, but particularly Example 1, wherein the valve of the implantable device comprises or is formed of one or more of: a polymer, a biomaterial, or a textile.

Example 30. The system of any the preceding examples, but particularly Example 1, wherein the valve is hingedly connected to the outer frame.

Example 31. The system of any the preceding examples, but particularly Example 1, wherein the valve comprises a ball.

Example 32. The system of any the preceding examples, but particularly Example 1, wherein the blood vessel comprises a superior vena cava or an inferior vena cava.

Example 33. The system of any the preceding examples, but particularly Example 1, wherein the control element is disposed in a sheath and comprises a first control element portion and a second control element portion and the valve comprises a first valve portion and a second valve portion, wherein the first control element portion is coupled to the first valve portion and the second control element portion is coupled to the second valve portion.

Example 34. The system of any the preceding examples, but particularly Example 33, wherein the piston in the restricted blood flow position is configured to tension the first and second control element portions and retract the first and second control element portions into the sheath.

Example 35. The system of any the preceding examples, but particularly Example 33, wherein the piston in the unrestricted blood flow position is configured to relieve tension on the first and second control element portions and extend the first and second control element portions from the sheath.

Example 36. The system of any the preceding examples, but particularly Example 1, wherein the implantable device is configured to modulate a volume of blood flowing from a superior vena cava into a right atrium to decrease right atrial pressure.

Example 37. A method of restricting blood flow within a blood vessel, comprising using the system of any one of Examples 1-35 to restrict blood flow within the blood vessel.

Example 38. A method of treatment for a subject having one or both of: congestive heart failure or chronic kidney disease, comprising using the system of any one of Examples 1-35 to restrict blood flow within the blood vessel.

Example 39. An actuation device configured to regulate blood flow through an implantable device positioned in a blood vessel, the actuation device comprising: a pump fluidly connected to a reservoir; a chamber comprising a first portion and a second portion; a manifold fluidly connected to the pump, the reservoir, and the chamber, wherein the manifold comprises at least one port configured to fluidly connect the reservoir to the first portion of the chamber; and a piston coupled to a control element of an implantable device and configured to move between a restricted blood flow position and an unrestricted blood flow position within the chamber, wherein: the piston is configured to move to the restricted blood flow position when a fluid is pumped from the reservoir through the manifold into the first portion of the chamber, and the piston is configured to return to the unrestricted blood flow position when the fluid is evacuated from the first portion of the chamber.

Example 40. The actuation device of any the preceding examples, but particularly Example 39, wherein the fluid comprises a gas.

Example 41. The actuation device of any the preceding examples, but particularly Example 39, wherein the fluid comprises a liquid.

Example 42. The actuation device of any the preceding examples, but particularly Example 39, wherein the fluid in the reservoir is a rechargeable fluid.

Example 43. The actuation device of any the preceding examples, but particularly Example 39, wherein the pump comprises an electromechanical pump.

Example 44. The actuation device of any the preceding examples, but particularly Example 39, wherein the pump is configured to be activated by an anatomy of a user.

Example 45. The actuation device of any the preceding examples, but particularly Example 39, wherein the manifold comprises a second port fluidly connected to a second portion of the chamber, wherein the piston is configured to move to the unrestricted blood flow position when the fluid enters the second port from the reservoir through the manifold.

Example 46. The actuation device of any the preceding examples, but particularly Example 45, wherein the piston is further configured to move to an intermediate position, wherein the fluid evacuates the second portion of the chamber through the second port into the reservoir through the manifold.

Example 47. The actuation device of any the preceding examples, but particularly Example 39, wherein the manifold comprises a second port fluidly connected to a second portion of the chamber, wherein the fluid is configured to be evacuated from the second portion of the chamber through the second port when the piston is in the restricted blood flow position.

Example 48. The actuation device of any the preceding examples, but particularly Example 47, wherein the actuation device further comprises an input element configured to direct flow through the at least one port or the second port.

Example 49. The actuation device of any the preceding examples, but particularly Example 47, further comprising a second reservoir, wherein the fluid is configured to flow from the second portion of the chamber to the second reservoir.

Example 50. The actuation device of any the preceding examples, but particularly Example 49, wherein the fluid is configured to be pumped from the second reservoir to the reservoir.

Example 51. The actuation device of any the preceding examples, but particularly Example 49, wherein the second reservoir comprises a low-pressure reservoir.

Example 52. The actuation device of any the preceding examples, but particularly Example 39, wherein the fluid connection between the pump and the reservoir comprises a one-way valve configured to enable fluid flow from the pump to the reservoir.

Example 53. The actuation device of any the preceding examples, but particularly Example 52, wherein the one-way valve comprises a check valve.

Example 54. The actuation device of any the preceding examples, but particularly Example 39, wherein the fluid connection between the manifold and the pump comprises a one-way valve configured to enable the fluid to flow from the manifold to the pump.

Example 55. The actuation device of any the preceding examples, but particularly Example 54, wherein the one-way valve comprises a check valve.

Example 56. The actuation device of any the preceding examples, but particularly Example 39, wherein the at least one port further fluidly connects the first portion of the chamber to the pump through the manifold.

Example 57. The actuation device of any the preceding examples, but particularly Example 56, wherein the at least one port is fluidly connected to the pump through the manifold to evacuate the fluid from the first portion of the chamber thereby moving the piston to the unrestricted blood flow position.

Example 58. The actuation device of any the preceding examples, but particularly Example 56, wherein the piston comprises a spring piston, wherein the spring piston is configured to automatically return to the unrestricted blood flow position when the fluid is evacuated from the first portion of the chamber.

Example 59. The actuation device of any the preceding examples, but particularly Example 39, wherein the reservoir is a high-pressure reservoir.

Example 60. The actuation device of any the preceding examples, but particularly Example 39, wherein the reservoir is a high-volume reservoir.

Example 61. The actuation device of any the preceding examples, but particularly Example 39, wherein the actuation device further comprises a power source.

Example 62. The actuation device of any the preceding examples, but particularly Example 61, wherein the pump comprises an electromechanical pump such that the power source is configured to electrically power the pump.

Example 63. The actuation device of any the preceding examples, but particularly Example 39, wherein the blood vessel comprises a superior vena cava or an inferior vena cava.

Example 64. The actuation device of any the preceding examples, but particularly Example 39, further comprising the implantable device that comprises: an outer frame, a valve disposed in the outer frame, and a control element configured to manipulate the valve between an unrestricted blood flow state and a restricted blood flow state, wherein a first end of the control element is coupled to the valve and a second end of the control element is coupled to the piston.

Example 65. The actuation device of any the preceding examples, but particularly Example 64, wherein the outer frame of the implantable device comprises a stent.

Example 66. The actuation device of any the preceding examples, but particularly Example 64, wherein the implantable device further comprises an articulating arm, wherein the control element is disposed in the articulating arm.

Example 67. The actuation device of any the preceding examples, but particularly Example 66, wherein the control element is configured to actuate the articulating arm, thereby actuating the valve between the unrestricted blood flow state and the restricted blood flow state.

Example 68. The actuation device of any the preceding examples, but particularly Example 66, wherein the articulating arm comprises a plurality of laser cut joints.

Example 69. The actuation device of any the preceding examples, but particularly Example 66, wherein the articulating arm comprises a plurality of linked segments.

Example 70. The actuation device of any the preceding examples, but particularly Example 64, wherein the valve of the implantable device comprises or is formed of one or more of: a polymer, a biomaterial, or a textile.

Example 71. The actuation device of any the preceding examples, but particularly Example 64, wherein the valve is hingedly connected to the outer frame.

Example 72. The actuation device of any the preceding examples, but particularly Example 64, wherein the valve comprises a ball.

Example 73. The actuation device of any the preceding examples, but particularly Example 64, wherein the control element is disposed in a sheath and comprises a first control element and a second control element and the valve comprises a first valve portion and a second valve portion, wherein the first control element is coupled to the first valve portion and the second control element is coupled to the second valve portion.

Example 74. The actuation device of any the preceding examples, but particularly Example 73, wherein the piston in the restricted blood flow position is configured to tension the first and second control elements and retract the first and second control elements into the sheath.

Example 75. The actuation device of any the preceding examples, but particularly Example 73, wherein the piston in the unrestricted blood flow position is configured to relieve tension on the first and second control elements and extend the first and second control elements from the sheath.

Example 76. The actuation device of any the preceding examples, but particularly Example 64, wherein the implantable device is configured to modulate a volume of blood flowing from a superior vena cava into a right atrium to decrease right atrial pressure.

Example 77. A method of restricting blood flow within a blood vessel, comprising using the implantable device of any one of Examples 64-75 to restrict blood flow within the blood vessel.

Example 78. A method of treatment for a subject having one or both of: congestive heart failure or chronic kidney disease, comprising using the implantable device of any one of Examples 64-75 to restrict blood flow within the blood vessel.

Example 79. A system for modulating blood flow through a blood vessel, the system comprising: an implantable device comprising: an outer frame, a valve disposed in the outer frame, a control element coupled to the valve, wherein the control element is configured to manipulate the valve between an unrestricted blood flow state and a restricted blood flow state, and a sensor coupled to the implantable device; and an actuation device comprising: a linear actuator coupled to the control element, a power source, and a microcontroller electrically coupled to the power source, the linear actuator, and the sensor, wherein the linear actuator is configured to tension the control element to position the valve in the restricted blood flow state in response to a first pressure state sensed by the sensor, and wherein the linear actuator is configured to release tension in the control element to position the valve in the unrestricted blood flow state in response to a second pressure state sensed by the sensor.

Example 80. The system of any the preceding examples, but particularly Example 79, wherein the linear actuator is an electromechanical linear actuator comprising a first magnet configured to rotate a nut rotatably disposed on a lead screw, the nut being coupled to the control element.

Example 81. The system of any the preceding examples, but particularly Example 79, wherein the linear actuator comprises a pneumatic linear actuator comprising a piston coupled to the control element and configured to inject compressed gas to tension the control element to move the valve into the restricted blood flow state and vent compressed gas to release tension in the control element to move the valve to the unrestricted blood flow state.

Example 82. The system of any the preceding examples, but particularly Example 79, wherein the linear actuator comprises a hydraulic linear actuator comprising a piston coupled to the control element and configured to inject a liquid to tension the control element to move the valve into the restricted blood flow state and vent a liquid to release tension in the control element to move the valve to the unrestricted blood flow state.

Example 83. The system of any the preceding examples, but particularly Example 79, wherein the sensor comprises one of: a strain gauge, a piezoelectric sensor, a capacitance sensor, or a vacuum pressure sensor.

Example 84. The system of any the preceding examples, but particularly Example 79, wherein the first pressure state sensed by the sensor indicates an increase in pressure in a right atrium.

Example 85. The system of any the preceding examples, but particularly Example 79, wherein the second pressure state sensed by the sensor indicates a decrease in pressure in a right atrium.

Example 86. The system of any the preceding examples, but particularly Example 79, wherein the outer frame of the implantable device comprises a stent.

Example 87. The system of any the preceding examples, but particularly Example 79, wherein the implantable device further comprises an articulating arm, wherein the control element is disposed in the articulating arm.

Example 88. The system of any the preceding examples, but particularly Example 87, wherein the control element is configured to actuate the articulating arm, thereby actuating the valve between the unrestricted blood flow state and the restricted blood flow state.

Example 89. The system of any the preceding examples, but particularly Example 87, wherein the articulating arm comprises a plurality of laser cut joints.

Example 90. The system of any the preceding examples, but particularly Example 87, wherein the articulating arm comprises a plurality of linked segments.

Example 91. The system of any the preceding examples, but particularly Example 79, wherein the valve of the implantable device comprises or is formed of one or more of: a polymer, a biomaterial, or a textile.

Example 92. The system of any the preceding examples, but particularly Example 79, wherein the valve is hingedly connected to the outer frame.

Example 93. The system of any the preceding examples, but particularly Example 79, wherein the valve comprises a ball.

Example 94. The system of any the preceding examples, but particularly Example 79, wherein the control element is disposed in a sheath and comprises a first control element portion and a second control element portion and the valve comprises a first valve portion and a second valve portion, wherein the first control element portion is coupled to the first valve portion and the second control element portion is coupled to the second valve portion.

Example 95. The system of any the preceding examples, but particularly Example 94, wherein the linear actuator in the unrestricted blood flow state is configured to tension the first and second control element portions and retract the first and second control element portions into the sheath.

Example 96. The system of any the preceding examples, but particularly Example 94, wherein the linear actuator in the unrestricted blood flow state is configured to relieve tension on the first and second control element portions and extend the first and second control element portions from the sheath.

Example 97. The system of any the preceding examples, but particularly Example 79, wherein the blood vessel is a superior vena cava or an inferior vena cava.

Example 98. The system of any the preceding examples, but particularly Example 79, wherein the implantable device is configured to modulate a volume of blood flowing from a superior vena cava into a right atrium to decrease right atrial pressure.

Example 99. A method of restricting blood flow within a blood vessel, comprising using the implantable device of any one of Examples 79-97 to restrict blood flow within the blood vessel.

Example 100. A method of treatment for a subject having one or both of: congestive heart failure or chronic kidney disease, comprising using the implantable device of any one of Examples 79-97 to restrict blood flow within the blood vessel.

Example 101. A system for modulating blood flow through a blood vessel, the system comprising: an implanted actuation device comprising: a linear actuator coupled to a control element of an implantable device, and a first magnet configured to induce rotation of the linear actuator; and a control device communicatively coupled to the linear actuator, wherein the control device comprises: a second magnet configured to generate a changing magnetic field pole direction to cause rotation of the first magnet, a power source, and a microcontroller electrically coupled to the power source, the second magnet, and a sensor communicatively coupled to the implantable device, wherein, in response to a first pressure state sensed by the sensor, the microcontroller is configured to cause rotation of the second magnet in a first direction to induce rotation of the first magnet, thereby causing the linear actuator to tension the control element to position a valve of the implantable device in a restricted blood flow state, and wherein, in response to a second pressure state sensed by the sensor, the microcontroller is configured to cause rotation of the second magnet in a second direction to induce rotation of the first magnet, thereby causing the linear actuator to release tension in the control element to position the valve in an unrestricted blood flow state.

Example 102. The system of any the preceding examples, but particularly Example 101, wherein the control device is implanted.

Example 103. The system of any the preceding examples, but particularly Example 102, wherein the control device in implanted subcutaneously.

Example 104. The system of any the preceding examples, but particularly Example 101, wherein the control device is disposed external to a body of a user of the system.

Example 105. The system of any the preceding examples, but particularly Example 101, further comprising the implantable device comprising: an outer frame, a valve disposed in the outer frame, the control element coupled to the valve, wherein the control element is configured to manipulate the valve between the unrestricted blood flow state and the restricted blood flow state, and the sensor coupled to the implantable device.

Example 106. The system of any the preceding examples, but particularly Example 105, wherein the sensor comprises one of: a strain gauge, a piezoelectric sensor, a capacitance sensor, or a vacuum pressure sensor.

Example 107. The system of any the preceding examples, but particularly Example 105, wherein the outer frame of the implantable device comprises a stent.

Example 108. The system of any the preceding examples, but particularly Example 105, wherein the implantable device further comprises an articulating arm, wherein the control element is disposed in the articulating arm.

Example 109. The system of any the preceding examples, but particularly Example 108, wherein the control element is configured to actuate the articulating arm, thereby actuating the valve between the unrestricted blood flow state and the restricted blood flow state.

Example 110. The system of any the preceding examples, but particularly Example 108, wherein the articulating arm comprises a plurality of laser cut joints.

Example 111. The system of any the preceding examples, but particularly Example 108, wherein the articulating arm comprises a plurality of linked segments.

Example 112. The system of any the preceding examples, but particularly Example 105, wherein the valve of the implantable device comprises or is formed of one or more of: a polymer, a biomaterial, or a textile.

Example 113. The system of any the preceding examples, but particularly Example 105, wherein the valve is hingedly connected to the outer frame.

Example 114. The system of any the preceding examples, but particularly Example 105, wherein the valve comprises a ball.

Example 115. The system of any the preceding examples, but particularly Example 101, wherein the blood vessel comprises a superior vena cava or an inferior vena cava.

Example 116. The system of any the preceding examples, but particularly Example 101, wherein the control element is disposed in a sheath and comprises a first control element portion and a second control element portion and the valve comprises a first valve portion and a second valve portion, wherein the first control element portion is coupled to the first valve portion and the second control element portion is coupled to the second valve portion.

Example 117. The system of any the preceding examples, but particularly Example 116, wherein the linear actuator in the restricted blood flow state is configured to tension the first and second control element portions and retract the first and second control element portions into the sheath.

Example 118. The system of any the preceding examples, but particularly Example 116, wherein the linear actuator in the unrestricted blood flow state is configured to relieve tension on the first and second control elements and extend the first and second control elements from the sheath.

Example 119. The system of any the preceding examples, but particularly Example 101, wherein the power source comprises a battery or a wall outlet.

Example 120. The system of any the preceding examples, but particularly Example 101, wherein the first magnet is a permanent magnet, and the second magnet is a permanent magnet.

Example 121. The system of any the preceding examples, but particularly Example 101, wherein the first magnet is a permanent magnet, and the second magnet is an electromagnet.

Example 122. The system of any the preceding examples, but particularly Example 101, further comprising a repeater magnet positioned in between the implanted actuation device and the control device.

Example 123. The system of any the preceding examples, but particularly Example 122, wherein the repeater magnet is implanted.

Example 124. The system of any the preceding examples, but particularly Example 122, wherein the repeater magnet is implanted subcutaneously.

Example 125. The system of any the preceding examples, but particularly Example 122, wherein the repeater magnet is part of a repeater module, the repeater module comprising a second power source configured to power the repeater magnet.

Example 126. The system of any the preceding examples, but particularly Example 125, wherein the repeater magnet further comprises a motor configured to rotate the repeater magnet.

Example 127. The system of any the preceding examples, but particularly Example 101, wherein the first pressure state sensed by the sensor indicates an increase in pressure in a right atrium.

Example 128. The system of any the preceding examples, but particularly Example 101, wherein the second pressure state sensed by the sensor indicates a decrease in pressure in a right atrium.

Example 129. An implantable device configured to regulate blood flow in a blood vessel, the implantable device comprising: an outer frame; a valve disposed in the outer frame; and a control element coupled to the valve, wherein the control element is configured to manipulate the valve between an unrestricted blood flow state and a restricted blood flow state.

Example 130. The implantable device of any the preceding examples, but particularly Example 129, further comprising a sensor coupled to the outer frame.

Example 131. The implantable device of any the preceding examples, but particularly Example 130, wherein the sensor comprises one of: a strain gauge, a piezoelectric sensor, a capacitance sensor, or a vacuum pressure sensor.

Example 132. The implantable device of any the preceding examples, but particularly Example 129, wherein the outer frame of the implantable device comprises a stent.

Example 133. The implantable device of any the preceding examples, but particularly Example 129, wherein the implantable device further comprises an articulating arm, wherein the control element is disposed in the articulating arm.

Example 134. The implantable device of any the preceding examples, but particularly Example 133, wherein the control element is configured to actuate the articulating arm, thereby actuating the valve between the unrestricted blood flow state and the restricted blood flow state.

Example 135. The implantable device of any the preceding examples, but particularly Example 133, wherein the articulating arm comprises a plurality of laser cut joints.

Example 136. The implantable device of any the preceding examples, but particularly Example 133, wherein the articulating arm comprises a plurality of linked segments.

Example 137. The implantable device of any the preceding examples, but particularly Example 129, wherein the valve of the implantable device comprises or is formed of one or more of: a polymer, a biomaterial, or a textile.

Example 138. The implantable device of any the preceding examples, but particularly Example 129, wherein the valve is hingedly connected to the outer frame.

Example 139. The implantable device of any the preceding examples, but particularly Example 129, wherein the valve comprises a ball.

Example 140. The implantable device of any the preceding examples, but particularly Example 129, wherein the blood vessel comprises a superior vena cava or an inferior vena cava.

Example 141. The implantable device of any the preceding examples, but particularly Example 129, wherein the control element is disposed in a sheath and comprises a first control element portion and a second control element portion and the valve comprises a first valve portion and a second valve portion, wherein the first control element portion is coupled to the first valve portion and the second control element portion is coupled to the second valve portion.

Example 142. The implantable device of any the preceding examples, but particularly Example 141, wherein the first and second control element portions are configured to be retracted into the sheath by an actuation device to move the first and second valve portions to the restricted blood flow state.

Example 143. The implantable device of any the preceding examples, but particularly Example 141, wherein the first and second control element portions are configured to be extended from the sheath by an actuation device to relieve tension on the first and second control elements to move the first and second valve portions to the unrestricted blood flow state.

Example 144. The implantable device of any the preceding examples, but particularly Example 129, wherein the blood vessel is a superior vena cava.

Example 145. The implantable device of any the preceding examples, but particularly Example 144, wherein the implantable device is configured to be implanted in a patient having chronic kidney disease and chronic heart failure.

Example 146. The implantable device of any the preceding examples, but particularly Example 144, wherein the implantable device is configured to modulate a volume of blood flowing from the superior vena cava into a right atrium to decrease right atrial pressure.

Example 147. The implantable device of any the preceding examples, but particularly Example 129, wherein the implantable device is configured to modulate a volume of blood flowing from a superior vena cava into a right atrium to decrease right atrial pressure.

Example 148. A method of restricting blood flow within a blood vessel, comprising using the implantable device of any one of Examples 129-146 to restrict blood flow within the blood vessel.

Example 149. A method of treatment for a subject having one or both of: congestive heart failure or chronic kidney disease, comprising using the implantable device of any one of Examples 129-146 to restrict blood flow within the blood vessel.

What is claimed is:

1. A system for modulating blood flow through a blood vessel, the system comprising:
an implantable device comprising:
an outer frame,
a valve disposed in the outer frame,
a control element coupled to the valve, wherein the control element is configured to manipulate the valve between an unrestricted blood flow state and a restricted blood flow state, and
a sensor coupled to the implantable device;
a magnetic gear train, wherein at least one magnet of the magnetic gear train is external to the implantable device; and
an actuation device comprising:
a linear actuator coupled to the control element,
a power source, and
a microcontroller electrically coupled to the power source, the linear actuator, the at least one magnet, and the sensor,
wherein the linear actuator, responsive to rotation of the magnetic gear train, is configured to tension the control element to position the valve in the restricted blood flow state in response to a first pressure state sensed by the sensor, and
wherein the linear actuator, responsive to rotation of the magnetic gear train, is configured to release tension in the control element to position the valve in the unrestricted blood flow state in response to a second pressure state sensed by the sensor.

2. The system of claim 1, wherein the linear actuator is an electromechanical linear actuator responsive to the magnetic gear train, wherein a first magnet of the magnetic gear train is configured to rotate a nut rotatably disposed on a lead screw, the nut being coupled to the control element.

3. The system of claim 1, wherein the sensor comprises one of: a strain gauge, a piezoelectric sensor, a capacitance sensor, or a vacuum pressure sensor.

4. The system of claim 1, wherein the first pressure state sensed by the sensor indicates an increase in pressure in a right atrium, and the second pressure state sensed by the sensor indicates a decrease in pressure in a right atrium.

5. The system of claim 1, wherein the implantable device further comprises an articulating arm, wherein the control element is disposed in the articulating arm.

6. The system of claim 5, wherein the control element is configured to actuate the articulating arm, thereby actuating the valve between the unrestricted blood flow state and the restricted blood flow state.

7. The system of claim 1, wherein the control element is disposed in a sheath and comprises a first control element portion and a second control element portion and the valve comprises a first valve portion and a second valve portion, wherein the first control element portion is coupled to the first valve portion and the second control element portion is coupled to the second valve portion.

8. The system of claim 7, wherein the linear actuator in the restricted blood flow state is configured to tension the first and second control element portions and retract the first and second control element portions into the sheath, and wherein the linear actuator in the unrestricted blood flow state is configured to relieve tension on the first and second control element portions and extend the first and second control element portions from the sheath.

9. The system of claim 1, wherein the at least one magnet of the magnetic gear train is external to a user of the implantable device or implanted at a different location than a location of the implantable device.

10. A system for modulating blood flow through a blood vessel, the system comprising:
an implanted actuation device comprising:
a linear actuator coupled to a control element of an implantable device, and
a first magnet configured to induce rotation of the linear actuator; and
a control device communicatively coupled to the linear actuator, wherein the control device comprises:
a second magnet configured to generate a changing magnetic field pole direction to cause rotation of the first magnet, wherein the second magnet is external to the implanted actuation device,
a power source, and
a microcontroller electrically coupled to the power source, the second magnet, and a sensor communicatively coupled to the implantable device,
wherein, in response to a first pressure state sensed by the sensor, the microcontroller is configured to cause rotation of the second magnet in a first direction to induce rotation of the first magnet, thereby causing the linear actuator to tension the control element to position a valve of the implantable device in a restricted blood flow state, and
wherein, in response to a second pressure state sensed by the sensor, the microcontroller is configured to cause rotation of the second magnet in a second direction to induce rotation of the first magnet, thereby causing the linear actuator to release tension in the control element to position the valve in an unrestricted blood flow state.

11. The system of claim 10, wherein the control device is implanted or implanted subcutaneously.

12. The system of claim 10, wherein the control device is disposed external to a body of a user of the system.

13. The system of claim 10, further comprising the implantable device comprising:
an outer frame,
the valve disposed in the outer frame,
the control element coupled to the valve, wherein the control element is configured to manipulate the valve between the unrestricted blood flow state and the restricted blood flow state, and
the sensor coupled to the implantable device.

14. The system of claim 10, wherein the implantable device further comprises an articulating arm, wherein the control element is disposed in the articulating arm.

15. The system of claim 14, wherein the control element is configured to actuate the articulating arm, thereby actuating the valve between the unrestricted blood flow state and the restricted blood flow state.

16. The system of claim 10, wherein the first magnet is a first permanent magnet, and the second magnet is a second permanent magnet or an electromagnet.

17. The system of claim 10, further comprising a repeater magnet positioned in between the implanted actuation device and the control device.

18. The system of claim 17, wherein the repeater magnet is part of a repeater module, the repeater module comprising a second power source configured to power the repeater magnet, and a motor configured to rotate the repeater magnet.

19. The system of claim 10, wherein the first pressure state sensed by the sensor indicates an increase in pressure in a right atrium, and wherein the second pressure state sensed by the sensor indicates a decrease in pressure in a right atrium.

20. A system for modulating blood flow through a blood vessel, the system comprising:
an implantable device configured to modulate blood flow between an unrestricted blood flow state and a restricted blood flow state, wherein the implantable device comprises a valve;
one or more sensors;
at least one magnet positionable external to the implantable device; and
an actuation device comprising:
a linear actuator coupled to a control element of the implantable device,
a power source, and
a microcontroller electrically coupled to the power source and the linear actuator,
wherein the microcontroller is configured to:
receive measurements from the one or more sensors; and
activate, based on the received measurements and a magnetic field generated by the at least one magnet, the actuation device of the implantable device to adjust the valve of the implantable device to control the blood flow.
